(19) United States
(12) Patent Application Publication
(10) Pub. No.: US 2024/0024657 A1
(43) Pub. Date: Jan. 25, 2024

(54) CIRCULATORY SUPPORT DEVICES AND METHODS

(71) Applicant: NXT Biomedical, LLC, Irvine, CA (US)

(72) Inventors: Robert C. Taft, Irvine, CA (US); Alexander Siegel, Irvine, CA (US); Glen Rabito, Irvine, CA (US); Joseph Passman, Irvine, CA (US); Stanton J. Rowe, Irvine, CA (US); Elliot Howard, Irvine, CA (US); Jeremy Koehler, Irvine, CA (US); Robert S. Schwartz, Irvine, CA (US); Abubaker Khalifa, Irvine, CA (US)

(73) Assignee: NXT Biomedical, LLC, Irvine, CA (US)

(21) Appl. No.: 18/256,914

(22) PCT Filed: Dec. 9, 2021
(86) PCT No.: PCT/US2021/072841

§ 371 Date: Jun. 9, 2023

Related U.S. Application Data
(60) Provisional application No. 63/171,946, filed on Apr. 7, 2021.
(60) Provisional application No. 63/135,978, filed on Jan. 11, 2021.
(60) Provisional application No. 63/123,401, filed on Dec. 9, 2020.

(51) Int. Cl. A61M 60/139; A61M 25/10; A61M 60/861; A61M 60/896

(52) U.S. Cl. CPC A61M 60/139; A61M 25/1011; A61M 25/1002; A61M 60/861; A61M 60/896; A61M 2025/1052; A61M 2025/1061

(57) ABSTRACT

Described herein are improved blood pumping devices, including improved intra-aortic balloon pumps and ventricular assist devices. The pumping efficiency of either device may be improved with the use of one or more valves, counter pulsation balloons, non-compliant tubular structures, secondary pumping balloons, and similar components.

### RELATED APPLICATIONS

This application claims benefit of and priority to U.S. Provisional Application Ser. No. 63/123,401 filed Dec. 9, 2020 entitled Circulatory Support Devices and Methods, U.S. Provisional Application Ser. No. 63/135,978 filed Jan. 11, 2021 entitled Circulatory Support Devices and Methods, U.S. Provisional Application Ser. No. 63/171,946 filed Apr. 7, 2021 entitled Circulatory Support Devices and Methods all of which are hereby incorporated herein by reference in their entireties.

### BACKGROUND OF THE INVENTION

Intra-aortic balloon pumps (IABP) are devices that help increase the amount and the ease of which a heart can pump blood into a patient's arteries. These pumps are often used on patients with cardiogenic shock, which can be caused by acute myocardial infarction cardiogenic shock (AMICS), Acute decompensated heart failure (ADHF), or similar conditions. Typically, Intra-aortic balloon pumps comprise a catheter with an inflatable balloon at its distal end. The balloon is inserted into the aorta and the balloon is configured to inflate when the heart relaxes during its pumping cycle, pushing blood flow back towards the coronary arteries. The balloon continues to cycle between inflation and deflation, augmenting the natural pumping action of the heart until the pump is removed from the patient.

However, the pumping cycles of intra-aortic balloon pumps are often unable to achieve clinically significant cardiac output improvement. In other words, the pumping is often not enough to provide a significant improvement to overcome the dysfunction of the patient's heart.

While a reduction in afterload can increase stroke volume via the Starling Effect, feedback from physicians indicates that the amount of stroke volume increase by an IABP is not enough. In patients that need a boost in cardiac output, some physicians turn to more expensive solutions with higher complications rates, such as Impella, VA Ecmo, and similar devices. Despite the higher costs, these alternative treatments have failed to show significant mortality benefit.

The previously described Impella and VA Ecmo are generally known as ventricular assist devices (VAD), also known as a mechanical circulatory support device, which are implantable mechanical pumps that helps pump blood from the ventricles of a patient's heart to the rest of the body.

The Impella device is widely used by physicians and typically includes a motorized impeller within a passage at the distal end of its catheter body. When activated, the impeller draws in blood from an inlet into the passage and pushes it out an outlet at a proximal location. Although an Impella device can be placed in the left, right or both ventricles of a heart, it is most frequently used in the left ventricle where the inlet is positioned in the left ventricle and the outlet is positioned on the other side of the aortic valve 22, within the ascending aorta or aortic arch.

However, Impella devices and similar ventricular assist devices are significantly more invasive than IABPs, with higher complication rates including bleeding and hemolysis and also tend to be more expensive. In addition, it may be desirable to improve offloading of the left ventricle when using an Impella device by further reducing myocardial oxygen demand.

Hence, what is needed are safer, cheaper, and improved devices and techniques for significantly improving cardiac output.

### SUMMARY OF THE INVENTION

Some embodiments of this specification are directed to intra-aortic balloon pumps that are configured to inflate and deflate at various times during a cardiac cycle to increase blood flow within a patient.

In some embodiments, the intra-aortic balloon pump includes one or more valves positioned near the pumping balloon of the intra-aortic balloon pump. For example, the intra-aortic balloon pump may include one valve located either proximally or distally of the pumping balloon. In another example, the intra-aortic balloon pump includes two valves located proximally and distally of the pumping balloon.

In some embodiments, the one or more valves are one-way valves configured to allow blood flow in only one direction, such as antegrade or proximally relative to the intra-aortic balloon pump. In some embodiments, the one or more valves are opened and closed by a control device (e.g., that causes inflation or deflation of occlusion balloons that comprise the one or more valves).

In some embodiments, a relatively non-compliant tubular structure can be positioned around the balloon and expanded against the patient's aorta to reduce compliance of the vessel. This reduced compliance may increase pumping efficiency.

In some embodiments, the distal valve may be configured to allow some retrograde blood flow during a cardiac cycle to help allow blood flow into vessels connecting to the ascending aorta and aortic arch.

In some embodiments, a second pumping balloon is included in the aortic arch or ascending aorta to help supply blood to vessels connected in this area.

In some embodiments, the one or more valves and the pumping balloon are connected and positioned on the same catheter body. In other embodiments, the one or more valves and the pumping balloon are connected and positioned on separate catheter bodies.

In some embodiments, the pumping balloon may include a structure to further bias or force it to a deflated configuration to decrease deflation speed. This structure can include elastic bands or shape memory mesh.

Some embodiments of this specification are directed to a ventricular assist device and/or devices that are used with a ventricular assist device.

In some embodiments, a catheter may include a counter pulsation balloon configured to inflate and deflate during a cardiac cycle within a left ventricle. The counter pulsation balloon may be located on a catheter body used solely for counter pulsation balloon or may be included on a catheter with other functionality, such as a ventricular assist device or intra-aortic balloon pump. If the counter pulsation balloon is located on its own dedicated catheter, it can be used alone or with other “off-the-shelf” catheters such as a ventricular assist device (e.g., Impella) or an intra-aortic blood pump catheter. In embodiments with separate catheters, the counter pulsation balloon catheter may include a passage configured for the second catheter (e.g., ventricular assist device or IABP catheter) to pass into.

The counter pulsation balloon may be a traditional balloon inflatable with gas or liquid, or may be a expanded via a mechanical scaffold structure underneath a balloon layer. The counter pulsation balloon may expand radially and generally symmetrically relative to an axis of its catheter, or may expand in a radially offset, asymmetrical manner relative to the axis of its catheter. The counter pulsation balloon may alternately expand in a generally linear or directional manner.

In some embodiments, any of the ventricular assist devices may further include features of the intra-aortic balloon pumps described in this specification. Alternately, any of the ventricular assist devices may be used with separate intra-aortic balloon pumps (i.e., on different catheter bodies instead of on the same catheter body).

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a view of an intra-aortic balloon pump.

FIG. 2 is a view of an intra-aortic balloon pump.

FIG. 3 is a view of an intra-aortic balloon pump.

FIG. 4 is a view of an intra-aortic balloon pump.

FIG. 5 is a view of a valve for an intra-aortic balloon pump.

FIG. 6 is a view of the valve of FIG. 5.

FIG. 7 is a view of the valve of FIG. 5.

FIG. 8 is a view of a valve for an intra-aortic balloon pump.

FIG. 9 is a view of the valve of FIG. 8.

FIG. 10 is a view of a valve for an intra-aortic balloon pump.

FIG. 11 is a view of the valve of FIG. 10.

FIG. 12 is a view of an intra-aortic balloon pump.

FIG. 13 is a view of an intra-aortic balloon pump.

FIG. 14 is a view of an intra-aortic balloon pump.

FIG. 15 is a view of the intra-aortic balloon pump of FIG. 14.

FIG. 16 is a view of an intra-aortic balloon pump.

FIG. 17 is a view of the intra-aortic balloon pump of FIG. 16.

FIG. 18 is a view of an intra-aortic balloon pump.

FIG. 19 is a view of a valve device.

FIG. 20 is a view of the valve device of FIG. 19.

FIG. 21 is a view of a valve device.

FIG. 22 is a view of a valve device.

FIG. 23 is a view of a valve device.

FIG. 24 is a view of the valve device of FIG. 23.

FIG. 25 is a view of the valve device of FIG. 23.

FIG. 26 is a view of a valve device.

FIG. 27 is a view of the valve device of FIG. 27.

FIG. 28 is a view of a valve device.

FIG. 29 is a view of a graph showing balloon inflation and deflation time.

FIG. 30 is a view of an intra-aortic balloon pump.

FIG. 31 is a view of a valve device.

FIG. 32 is a view of an intra-aortic balloon pump.

FIG. 33 is a view of a ventricular assist device.

FIG. 34 is a view of a ventricular assist device.

FIG. 35 is a view of the ventricular assist device of FIG. 34.

FIG. 36 is a view of a graph showing pressure changes with various treatment devices.

FIG. 37 is a view of a ventricular assist device.

FIG. 38 is a view of the ventricular assist device of FIG. 37.

FIG. 39 is a view of a ventricular assist device.

FIG. 40 is a view of the ventricular assist device of FIG. 39.

FIG. 41 is a view of a ventricular assist device.

FIG. 42 is a view of the ventricular assist device of FIG. 41.

FIG. 43 is a view of a scaffold for a ventricular assist device.

FIG. 44 is a view of the scaffold of FIG. 43.

FIG. 45 is a view of the scaffold of FIG. 43.

FIG. 46 is a view of a ventricular assist device.

FIG. 47 is a view of a ventricular assist device.

FIG. 48 is a view of a ventricular assist device.

FIG. 49 is a view of a ventricular assist device.

FIG. 50 is a view of a ventricular assist device.

FIG. 51 is a view of a ventricular assist device.

FIG. 52 is a view of a ventricular assist device.

FIG. 53 is a view of catheter with a counter pulsation balloon and an intra-aortic balloon.

FIG. 54 is a view of a counter pulsation balloon catheter and an intra-aortic balloon catheter.

FIG. 55 is a view of an adapter for a counter pulsation balloon catheter and an intra-aortic balloon catheter.

FIG. 56 is a view of an adapter for a counter pulsation balloon catheter and an intra-aortic balloon catheter.

FIG. 57 is a view of a counter pulsation balloon catheter.

FIG. 58 is a view of an intra-aortic balloon catheter.

FIG. 59 is a view of an intra-aortic balloon catheter.

FIG. 60 is a view of a counter pulsation balloon catheter.

FIG. 61 is a view of a counter pulsation balloon catheter.

FIG. 62 is a view of a mechanical ventricular assist device.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The embodiments of the present specification are directed to devices and methods of increasing the amount and the ease of which a heart can pump blood into a patient's arteries. Generally, this may be accomplished with various improvements on traditional intra-aortic balloon pump designs and ventricular assist devices, which are two specific types of blood pumps used to improve blood pumping in a patient. For this reason, the term blood pump or catheter-based blood pump in this specification encompasses intra-aortic balloon pumps in which a pumping mechanism is positioned within an aorta (or similar location), ventricular assist devices in which a pumping mechanism is at least partially positioned within a left ventricle of a heart, and pumping devices used at other locations beyond the heart or aorta.

While the term intra-aortic balloon pump is generally described in this specification as being used within an aorta of a patient, it should be understood that other, non-aortic locations may also be used. Hence, this term should not be considered to strictly limit these embodiments to only use within an aorta. Similarly, the term ventricular assist devices are used in this specification as being used within a left ventricle of a patient, it should be understood that other, non-ventricular locations may also be used. Hence, this term should not be considered to strictly limit theses embodiments to only use within a left ventricle.

Generally, embodiments of intra-aortic balloon pumps are described first in this specification while embodiments of ventricular assist devices are discussed afterwards.

Currently used intra-aortic balloon pumps typically include an inflatable balloon to increase cardiac output from the heart and also increase the supply of oxygen to the heart. The balloon is typically positioned in the aorta (e.g., descending aorta) and as the heart ventricles contract and release the blood (systole), the intra-aortic balloon pump deflates and thereby reduces resistance and increases antegrade blood flow. As the ventricles of the heart relax (diastole) and fills with blood again, the balloon quickly expands and thereby increases the flow of blood to the coronary arteries (the arteries that supply O2 to the heart). Combining these actions reduces the heart's need for oxygen and improves the O2 delivery to the heart.

Currently used intra-aortic balloon pumps typically include an elongated balloon that is fixed at the distal end of an elongated catheter body. The catheter includes a passage in communication with an interior of the balloon and to a pumping mechanism that is configured to rapidly inflate and deflate the balloon within an aorta (e.g., descending thoracic aorta and/or aortic arch).

The catheter is connected to a IABP control device that is configured to rapidly inflate and deflate the balloon, often with a gas such as helium, via a pump mechanism. The IABP control console also typically uses an electrocardiogram (e.g., via ECG leads) to measure heart activity and a blood pressure transducer (e.g., on or near the catheter) to measure blood pressure. These values are used by the IABP control device to determine the correct timing for inflating and deflating the balloon.

Some of the blood pump embodiments discussed herein are directed to an intra-aortic balloon pump with one or more valves located near a proximal and/or a distal end of the balloon. These valves can improve cardiac output significantly compared to use of the balloon alone. Additionally, the use of the one or more valves can be a less expensive and less complicated approach over alternative pumps, such as Impella or VA Ecmo.

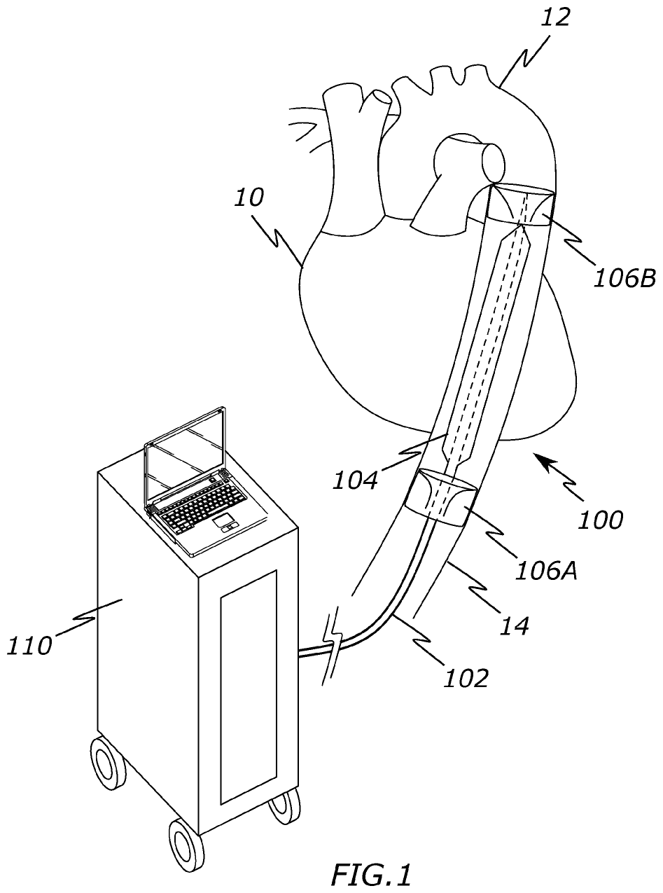

FIG. 1 illustrates one embodiment of an intra-aortic balloon pump 100 having one or more valves 106 to increase the pumping efficiency of a pumping balloon 104. While either a proximal valve 106A or a distal valve 106B may be included, including both valves 106A, 106B may result in greater efficiency of the pumping cycles of the pump 100.

Generally, the intra-aortic balloon pump 100 comprises an elongated catheter body 102 (e.g., 7-12 Fr) having an elongated pumping balloon 104 fixed near a distal end of the body 102. For example, the pumping balloon 104 may have an inflated volume of within an inclusive range of about 2.5 to 50 mL, may have a length within the inclusive range of about 20 to 54 cm, and an inflated diameter within the inclusive range of about 12-20 mm.

The catheter body 102 has one or more inflation ports that are in communication with an inflation passage within the body 102 that extends from the distal location of the balloon 104 to a proximal end of the body 102. The proximal end of the body 102 is in communication with a IABP control device 110 (e.g., via a tube and luer connection) which allows an inflation media (e.g., a gas such as helium) to quickly inflate the balloon 104 via the inflation passage of the body 102. The control device 110 may also be referred to as an inflation control device and may control inflation generally but also may control inflation relative to heart measurements that help determine a patient's cardiac cycle.

In some embodiments, the valves 106A, 106B may be one-way valves that open and allow passage of blood in one direction but close and prevent passage of blood moving in a second direction. In this respect, the valves 106A, 106B may be passively opened and closed by the blood flow itself.

For example, such a one-way valve may have one or more flaps similar to heart valve flaps. These one or more flaps may be composed of a flexible material that are generally restricted in movement in one axial direction of the catheter body 102 but allows to bend open in a second axial direction of the catheter body 102.

In other embodiments, the valves 106A, 106B may be actively controlled to open and close at a desired time. For example, one or more balloons can be used as a valve 106A, 106B, allowing a computerized device (e.g., the IABP control device 110) to inflate and close off the descending aorta 14 at the desired time.

Additional examples of different passively-controlled and actively-controlled valve types and configurations will be discussed in greater detail later in this specification.

While a proximal valve 106A and a distal valve 106B are both shown, an alternate embodiment may include only one of these valves.

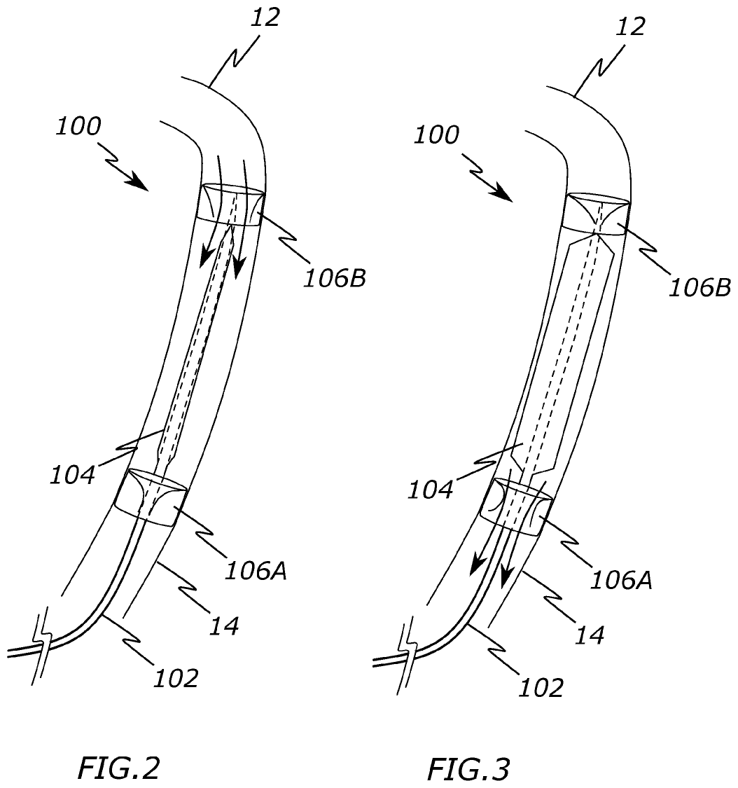

FIGS. 2 and 3 illustrate one possible mode of operation of the intra-aortic balloon pump 100. First, the distal end of the intra-aortic balloon pump is positioned within a location of a patient's descending aorta 14. The IABP control device 110 is activated and any ECG leads and/or pressure transducers are positioned on/in the patient and connected to the IABP control device 110 as necessary. In FIG. 2, the pumping balloon 104 is in a generally deflated state, meaning most or all of the inflation media has been removed from its interior so that it occupies a reduced cross-sectional diameter size (e.g., similar to the catheter body 102).

As the pumping balloon 104 achieves its reduced diameter, deflated state, its size reduction tends to pull blood towards it. In the case of passively-controlled one-way valves, the valves 106A, 106B are both configured to open with blood flow or pressure away from the aortic arch 14 and heart 10 (i.e., antegrade), but close with blood flow or pressure towards the aortic arch 14 and heart 10 (i.e., retrograde). Hence, the proximal valve 106A may achieve a closed configuration and the distal valve 106B may achieve an open configuration. This allows blood from the heart 10 and aortic arch 14 to be pulled into an area adjacent to the pumping balloon 104 and in between the two valves 106A, 106B (assuming two valves are used). However, little, if any, blood from a location proximal of the proximal valve 106A is brought into this space.

Turning to FIG. 3, the pumping balloon 104 is quickly inflated, which displaces the blood that was previously surrounding the pumping balloon 104. The movement of this blood retrograde towards the heart 10, or more accurately the retrograde pressure gradient of the blood across the distal valve 106B, will cause the distal valve 106B to close, preventing most or all of that blood from moving base the distal valve 106B. Additionally, this blood displacement or pressure gradient across the proximal valve 106A pushes against the proximal valve 106A, causing it to open and most, if not all, of the blood to move proximally and antegrade away from the heart 10.

Generally, the pumping balloon 104 will be deflated when the heart ventricles contract and release blood (systole) and is inflated when the ventricles of the heart relax (diastole). The timing of this inflation and deflation can be monitored and controlled by the IABP control device 110, which may be monitoring the patient's heart cycle via ECG and/or via blood pressure (e.g., via a pressure transducer in or near the intra-aortic balloon pump 100).

In the case of valves 106A, 106B that are actively controlled (e.g., that each have a separately inflatable occlusion balloon), the cycle may be similar to that previously described, except that the intra-aortic balloon pump 100 may cause the occlusion balloons of the valves 106A, 106B to open and close at the desired times instead of the blood flow from the pumping balloon 104 causing the one-way valves to open and close.

Specifically, in FIG. 2, the proximal valve 106A is closed (e.g., inflated) by the IABP control device 110, the distal valve 106B is opened (e.g., deflated) by the IABP control device 110, and the pumping balloon 104 is then deflated. In FIG. 3, the proximal valve 106A is opened (e.g., deflated) by the IABP control device 110, the distal valve 106B is closed (e.g., inflated) by the IABP control device 110, and the pumping balloon 104 is then inflated. In either scenario, the proximal valve 106A and distal valve 106B may be opened or closed just prior to the inflation state of the pumping balloon 104 shown in either figure. In other words, the open or closed state of the valves should be achieved first and then the pumping balloon 104 adjusted to a desired inflation state.

In such embodiments with actively-controlled valves 106A, 106b, the IABP control device 110 may include software configured to perform the previous valve closure and pumping balloon 104 inflation sequence. Hence, the IABP control device 110 may include a processor configured to execute software, memory configured to store software and be read by the processor, a display configured to output various sensor and control data, and input controls configured to allow controls of various aspects of the IABP control device 110.

In the case of using individual occlusion balloons in each valve 106A, 106B, the intra-aortic balloon pump 100 may include separate inflation passages in the catheter body 102 in communication with each occlusion balloon of each valve 106A, 106B, as well as the appropriate multichannel tubing to allow separate inflation media communication with the IABP control device 110. However, it may also be possible to configure the balloon pump 100 so that only a single inflation passages between the IABP control device 110 and catheter body 102 can inflate all balloons in the correct order. For example, the inflation passage of the catheter body 102 may have one or more one-way valves (e.g., two valves) that selectively allow inflation media into the balloons in the desired inflation order for each inflation/deflation cycle.

The intra-aortic balloon pump 100, as well as other pump embodiments discussed in this specification, may have several advantages over prior balloon pumps, impeller devices, or other pump mechanisms. First, such a device may be less expensive to manufacture than impeller-type pump devices. Further, such an intra-aortic balloon pump 100 may have reduced complications compared to an impeller-type pump devices, especially since it can have a lower delivery profile (e.g., less than 10 Fr). It may also provide additional cardiac output than traditional intra-aortic balloon pumps, so that even more blood flow is targeted at the kidneys to improve renal perfusion and urine output so as to help break the cardiorenal determination cycle that can sometimes occur.

Additional variations and embodiments of the intra-aortic balloon pump 100 are discussed further below. It should be emphasized that while these embodiments may describe some alternate features, any of the features in any of the embodiments may be combined together. In other words, any of the different features of the embodiments of this specification can be mixed and matched with each other. Therefore, while specific embodiments are discussed and shown in the figures, these embodiments are not the only possible configurations specifically contemplated.

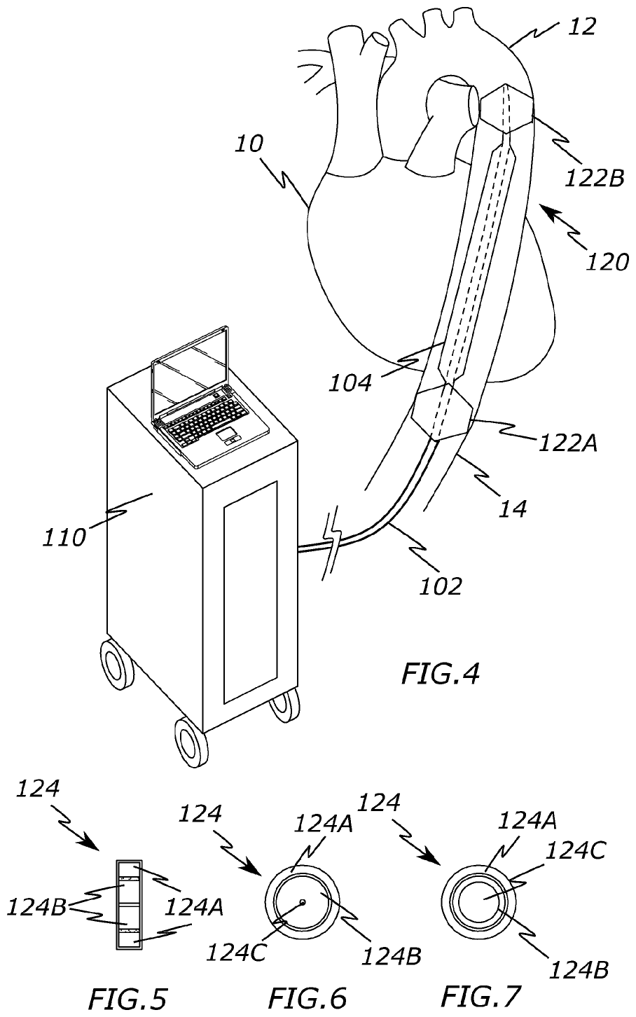

As previously discussed, balloons may be used as proximal and distal valves on an intra-aortic balloon pump. One such intra-aortic balloon pump 120 is illustrated in FIG. 4, including a proximal occlusive balloon 122A located at or beyond a proximal end of the pumping balloon 104, as well as a distal occlusive balloon 122B located at or beyond a distal end of the pumping balloon. Each occlusive balloon 122A, 122B are configured to expand to a size and shape sufficient to occlude a patient's descending aorta 14.

These occlusive balloons 122A, 122B can be configured to be used as discussed for FIGS. 2 and 3. For example, the proximal occlusive balloon 122A is closed (e.g., fully inflated) by the IABP control device 110, the distal occlusive balloon 122B is opened (e.g., deflated) by the IABP control device 110, and the pumping balloon 104 is then deflated. In the alternate cycle, the proximal occlusive balloon 122A is opened (e.g., deflated) by the IABP control device 110, the distal occlusive device 122B is closed (e.g., fully inflated) by the IABP control device 110, and the pumping balloon 104 is then inflated. In either scenario, the proximal occlusive balloon 122A and distal occlusive balloon 122B may be opened or closed just prior to the inflation state of the pumping balloon 104 discussed. In other words, the open or closed state of the occlusive balloons should typically be achieved first and then the pumping balloon 104 adjusted to a desired inflation state.

The occlusive balloons 122A, 122B can be configured for the desired inflation sequence in several different ways. First, three different inflation passages may be included between the IABP control device 110 and the balloons 122A, 122B, and 104 so that each of the balloons have their own isolated passage. These passages can extend through the catheter body 102 and any connective tubing connected to the IABP control device 110. Hence, the IABP control device 110 can deliver or remove the desired amount of inflation media to the interior of each balloon 122A, 122B, 104 at the desired time to complete the intended inflation sequence.

Since it can be desirable to maintain a small cross sectional catheter diameter size, it is also possible to use less than three inflation lumens. For example, two inflation lumens may be used. In such a configuration, a first inflation lumen may be connected to and in communication with the distal occlusive balloon 122B and the pumping balloon 104, while the second inflation lumen may be connected to and in communication with the proximal occlusive balloon 122B. Hence, the distal occlusive balloon 122B and the pumping balloon 104 can be inflated or deflated at roughly the same time and therefore separately controlled from the proximal occlusive balloon 122B.

Since the distal occlusive balloon 122B may generally have a smaller volume than the pumping balloon 104, it may tend to fully inflate or deflate much quicker than the pumping balloon 104, which may be desirable. However, it may be possible to increase this inflation differential/speed by increasing the inflation port size or number within the proximal occlusive balloon 122B as compared with the pumping balloon 104.

In another embodiment, only a single inflation passage may be used to inflate and deflate the balloons 122A, 122B, 104 in the desired sequences. In one example, this may be achieved by including a distal occlusive balloon 122B that has a different inflated shape than the proximal occlusive balloon 122A, such that one is generally open when inflated and the other is generally closed when inflated, and vice versa. Such balloon designs are discussed in further detail later in this specification.

The occlusive balloons 122A, 122B may have a variety of different shapes and configurations. Both occlusive balloons 122A, 122B may be almost or entirely identical or they may have different shapes and/or structures.

In one embodiment, one or more of the occlusive balloons 122A, 122B may be a single lumen balloon. Such a single lumen balloon may be formed, in one example, by bonding the edges of a tube of material over an inflation port and to the outer wall (or optionally within layers of the wall) of the catheter body 102.

Alternately, each balloon 122A, 122B may be composed of multiple balloon segments. In one embodiment, these balloon segments partially extend around the circumference of the catheter body 102 like slices of a pie so that together they all encircle the catheter body 102. Each balloon forms their own lumen and have their own inflation port into the inflation passage of the catheter body 102. Any number of balloon segments are possible, such as 2, 3, 4, 5, 6 or more.

In another embodiment seen in FIGS. 5, 6, and 7, a multi-segment balloon 124 is illustrated as having an outer tubular balloon segment 124A that is separated from and separately inflated from an inner tubular balloon segment 124B (note, FIG. 5 is a side cross sectional view of the balloon 124 while FIGS. 5 and 6 are views looking along the axis of the catheter body 102). The outer tubular balloon segment 124A can be inflated to help anchor the catheter body 102 in place and therefore remains inflated until the catheter 120 is removed by a physician. The inner tubular balloon segment 124B has a chamber that is separate from the outer tubular balloon segment 124A and therefore can be inflated or deflated as desired by the IABP control device 110. This inflation or deflation increases the size of the lumen 124C within the inner tubular balloon segment 124B, thereby opening or closing the lumen 124C. In some configurations, it may be helpful to compose the outer tubular balloon segment 124A of stiffer or less compliant material while the inner balloon segment 124B is composed of more compliant material.

As previously discussed, in some embodiments it may be desirable to use only a single inflation lumen within the catheter body 102. One way this may be accomplished is by including proximal and distal occlusion balloons that each have different shapes when inflated. For example, the distal occlusion balloon 122B may inflate to occlude the descending aorta 14 and deflate to open the descending aorta 14, but the proximal occlusion balloon 122B may achieve an open state of the descending aorta 14 when inflated and occlude the descending aorta 14 when deflated. However, such a balloon can be used in either the proximal or distal location.

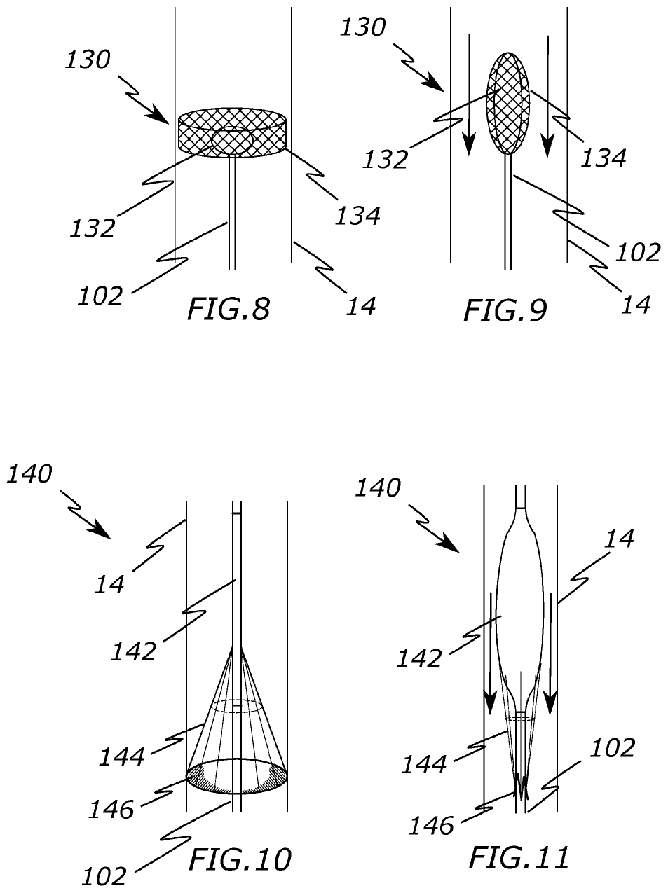

FIGS. 8 and 9 illustrate one embodiment of an occlusion balloon assembly 130 that opens the descending aorta 14 when inflated and occludes the descending aorta 14 when deflated. The occlusion balloon 130 includes a scaffold 134 that mostly or entirely surrounds a balloon 132. The scaffold 134 may have a native or unrestrained shape that radially expands to about the inner diameter of an descending aorta 14. This can be a disc shape, cylindrical shape, spherical shape, or similar shapes. Hence, when the balloon 132 within the scaffold 134 is mostly or fully deflated, as seen in FIG. 8, the scaffold 134 blocks the descending aorta 14. However, when the balloon 132 is inflated, it is configured to expand axially and therefore temporarily deform the scaffold 134 to a radially smaller shape (e.g., an oval shape), thereby allowing blood to flow around the scaffold 134.

The scaffold 134 can be composed of a rigid but deformable structure. For example, the structure may be composed of a shape memory material such as Nitinol. The structure may be formed from braided wires to form a mesh structure in a desired shape or can be laser cut from another structure such as a cylinder. The scaffold 134 may also be heat set to help bias it to its desired occlusive shape when not pushed on by the balloon 132.

The balloon 132 may expand in an oval or elongated shape with its longest dimension generally aligned with the axis of the catheter body 102. In one example, this can be achieved by using a noncompliant material on part or all of the balloon 132 that restricts its inflated shape. However, depending on the shape of the scaffold 134, the scaffold 134 may help force itself and the balloon 132 to the elongated shape.

FIGS. 10 and 11 illustrate another embodiment of an occlusion balloon assembly 140 that opens the descending aorta 14 when inflated and occludes the descending aorta 14 when deflated. A scaffold 144 is connected to a balloon 142 at its first end and is connected to a flexible barrier 146 at its second end. When the balloon 142 is deflated to a smaller diameter, as seen in FIG. 10, the first end of the scaffold 144 is also reduced in diameter, which forces its second end to radially expand outward. Since the flexible barrier 146 is connected at the second end of the scaffold 146, it is expanded radially outward to form a generally flat planar shape that blocks most or all of the descending aorta 14.

When the balloon 142 is inflated to a larger diameter, as seen in FIG. 11, the first end of the scaffold 144 is also increased in diameter, which forces its second end to radially decrease in diameter. Since the flexible barrier 146 is connected at the second end of the scaffold 146, it radially decreases or folds inward against the catheter body 102 to open the descending aorta 14. The inflated size of the balloon 142 is such that, despite being inflated, it still allows substantial passage of blood around it.

In one example, the scaffold 144 may include a plurality of struts that extend between its first end and second end. The struts may generally form a lever by connecting or contacting a fulcrum, such as a raised area on the catheter body 102 between the first end and the second end of the scaffold 144.

In one example, the flexible barrier 146 may have a generally circular and planar shape. The flexible barrier 146 may also be composed of a material that can easily fold and unfold, such as a polymer sheet.

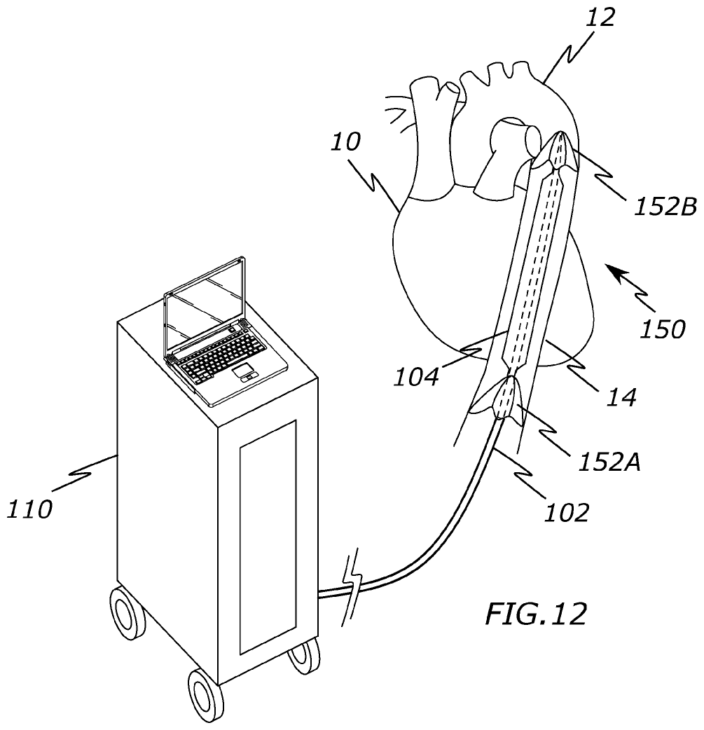

In another embodiment, the proximal and distal valves may be umbrella-style valves. For example, FIG. 12 illustrates another embodiment of an intra-aortic balloon pump 150 that is generally similar to the previously described intra-aortic balloon pump 100 but includes a proximal umbrella-style valve 152A located proximally of the pumping balloon 104 and a distal umbrella-style valve 152B located distally of the pumping balloon 104.

The valves 152A, 152B generally may be composed of a flexible material formed in a conical or concave shape that increases in diameter in the proximal direction (e.g., antegrade direction). Blood flowing proximally or antegrade causes the flexible material of the valves 152A, 152B to close or decrease in diameter, while blood flowing distally or retrograde pushes into the interior of the conical shape to radially expand the flexible material.

The flexible material may be a polymeric material or sheet configured in a conical shape radially around the catheter body 102. Optionally, a rigid scaffold may be included to help provide structure to the valves 152A, 152B. The scaffold may include a plurality of struts or braided mesh wires.

Hence, the valves 152A, 152B are passively opened and closed by the pressure gradient or flow direction of the blood in the descending aorta 14. Alternately, a small balloon may be included under the flexible material to help prop open the flexible material to ensure the valve closes.

In some embodiments, the pumping balloon 104 is directly exposed to the walls of the descending aorta 14. In other words, there is no other component between the pumping balloon 104 and the sidewalls of the descending aorta 14.

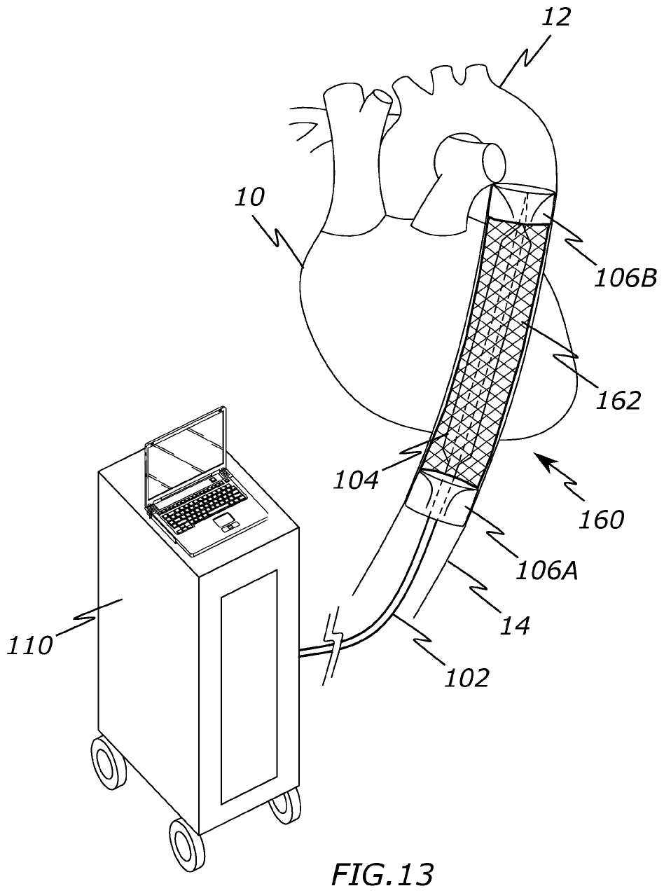

In some embodiments, as seen in FIG. 13, a tubular member 162 can be positioned or extend between the proximal valve 106A and the distal valve 106B of an intra-aortic balloon pump 160. The tubular member 162 may have increased stiffness or compliance as compared with the descending aorta 14, which may increase the pumping efficiency of the intra-aortic balloon pump 160, allowing it to pump more blood through the descending aorta 14.

The tubular member 162 may be comprised of a relatively stiff laser-cut stent (e.g., Nitinol or stainless), a braided stent with relatively large and stiff wires (e.g., Nitinol or stainless), a polymer stent, a tubular inflatable balloon, or similar structures that reduce aortic compliance.

This tubular member 162 can connect to inner ends of the valves 106A, 106B, or the valves 106A, 106B may be positioned inside the tubular member 162. Alternately, the increased pumping efficiency may allow only one of the valves 106A, 106B to be included. Alternately, the tubular member 162 may be used alone with the pumping balloon 104 without any valves. The increased efficiency may also allow for smaller pumping balloons 104 to be used or for a reduced number of inflation/deflation cycles.

It is possible that with some embodiments, the increased pumping efficiency may create some tendency to pull blood from vessels in the ascending aorta and aortic arch 12, such as the coronary arteries, brachiocephalic artery, left common carotid artery, and left subclavian artery. Hence, any of the embodiments of this specification may include one or more features, such as balloons or additional valves near these arteries to help prevent blood into these arteries from being reduced or even pulled out.

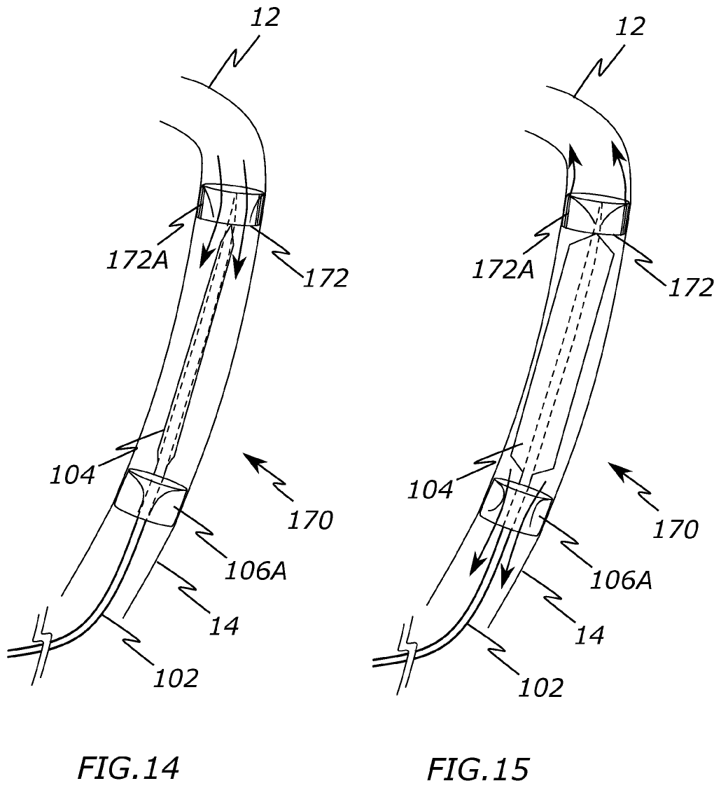

For example, in any of the embodiments in this specification, the distal valve may be created such that, in its closed or occluded state, it allows a small amount of blood to backflow in a retrograde direction into the aortic arch 12 and into the cerebral and coronary arteries. In other words, in its closed or occluded state, the valve is slightly leaky. FIGS. 14 and 15 illustrate one such embodiment of an intra-aortic balloon pump 170 that is generally similar to the previously described intra-aortic balloon pump 100 but includes a “leaky” distal valve 172.

The backflow or “leakiness” of the distal valve 172 may be achieved in several different ways. For example, one or more channels 172A can be included that always remain open. These channels may be located near or around the outer perimeter of the valve 172. Alternately, if the valve includes one or more leaflets, they can be shaped such that they leave a small gap in their closed state. Similarly, if an occlusion balloon is used, it may be configured with either channels or similar features along its outer surface such that it allows some blood flow through when in a closed or expanded state. In some examples, the valve 172 is configured to allow retrograde backflow of about 5%-40% of the blood passing through the valve 172, (e.g., 5%, 10%, 15%, 20%, 25%, 30%, 35%, 40%, or more).

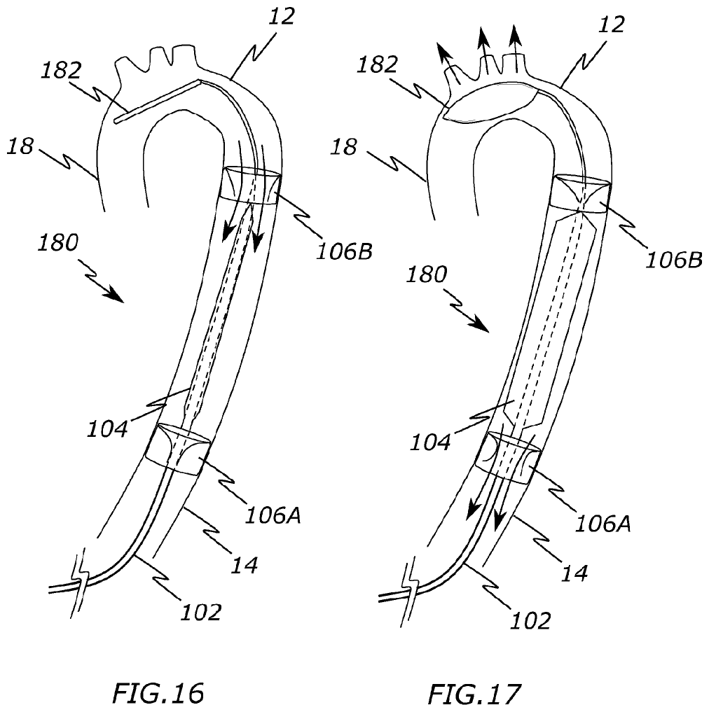

Any of the embodiments of this specification may also include an additional pumping balloon to also pump blood into the cerebral and coronary arteries. For example, FIGS. 16 and 17 illustrate an intra-aortic balloon pump 180 that is generally similar to the previously described pump 180, but further includes a distal pumping balloon 182.

The distal pumping balloon 182 may be located distally of the distal valve 106B and within the aortic arch 12 or the ascending aorta 18. As seen in FIG. 16, when the distal valve 106B is open and the pumping balloon 104 is deflated, the distal pumping balloon 182 is also deflated. As seen in FIG. 17, when the distal valve 106B closes and the pumping balloon 104 inflates, the distal pumping balloon 182 may also inflate, either simultaneously or close to the same time. This inflation of the distal pumping balloon 182 may push some of the blood within the ascending aorta 18 and aortic arch 12 into the vessels connected to it (e.g., the coronary arteries, brachiocephalic artery, left common carotid artery, and left subclavian artery).

The distal pumping balloon 182 may be connected on a distal extension/portion of the catheter body 102 and may have its own inflation passage for independent control, or may share the same inflation passage as the pumping balloon 104. Alternately, the distal pumping balloon 182 may be connected on its own separate catheter that can be moved independently of the catheter body 102, pumping balloon 104, and valves 106A, 106B.

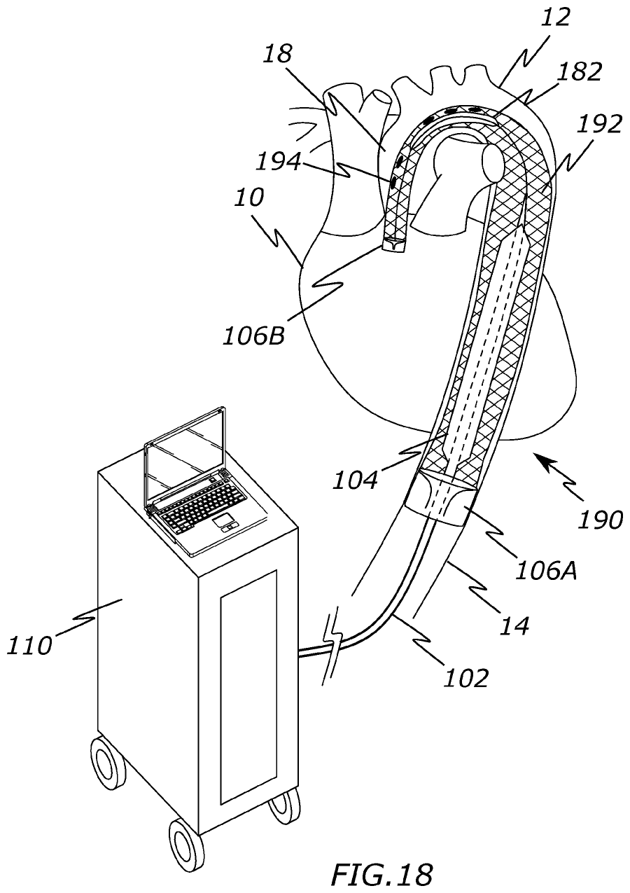

Any of the embodiments of this specification may also include a covered stent, sheath, or catheter that extends around the aortic arch 12 and into the ascending aorta 18 to help prevent any reduction in blood from reaching the cerebral and coronary arteries. Specifically, FIG. 18 illustrates an intra-aortic balloon pump 190 that is generally similar to the previously discussed intra-aortic balloon pump 100 but includes a covered stent 192 that extends from the proximal valve 106A, around the aortic arch 12, and at least into the ascending aorta 18.

The distal valve 106B may be positioned at and connected to the distal end of the covered stent 192 so as to selectively allow blood to enter the passage created by the covered stent 192. The distal portion of the covered stent 192 and the distal valve 106B may have a diameter that is smaller than the diameter of the ascending aorta 18, which allows some blood to pass around the covered stent and enter the cerebral and coronary arteries. However, this distal portion may taper to a larger diameter near or past the aortic arch 12 and within the descending aorta 14.

The covered stent 192 may include a scaffold and an outer blood impermeable layer on the scaffold. For example, the stent may be a braided or laser cut tubular structure that is composed of Nitinol or a similar alloy. The blood impermeable layer may be a polymer sleeve or similar material.

The covered stent 192 may terminate within the aortic arch 12 or the ascending aorta 18. In some embodiments, the covered stent 192 may terminate very close to the start of the ascending aorta 18, just above the aortic valve.

Optionally, the covered stent may include one or a plurality of relatively small one-way side valves 194 that are positioned within the sidewall of the covered stent 192 along the ascending aorta 18 and/or aortic arch 12. As pressure within the covered stent 192 increases, smaller amounts of blood may escape through the side valves 194 to supply the cerebral and coronary arteries. In one example, these sidewall valves 194 may be foil valves that allow blood to pass out of the covered stent 192 but not into it.

Optionally, the covered stent 192 may also include a distal pumping balloon 182, as discussed in prior embodiments. This distal pumping balloon 182 may help pump blood out of the sidewall valves 194 and into the cerebral and coronary arteries.

While some of the embodiments of this specification have been described in terms of both the pumping balloon 104 and the valves 106A, 106B (among other components) as being on the same catheter, it is also contemplated that the pumping balloon 104 can be separate from any valve and/or stent component. In this respect, currently available “off-the-shelf” intra-aortic balloon pumps can be used with a separate valve device. This allows either the valve device to be deployed first, followed by the intra-aortic balloon pump, or the intra-aortic balloon pump to be deployed first, followed by the valve device.

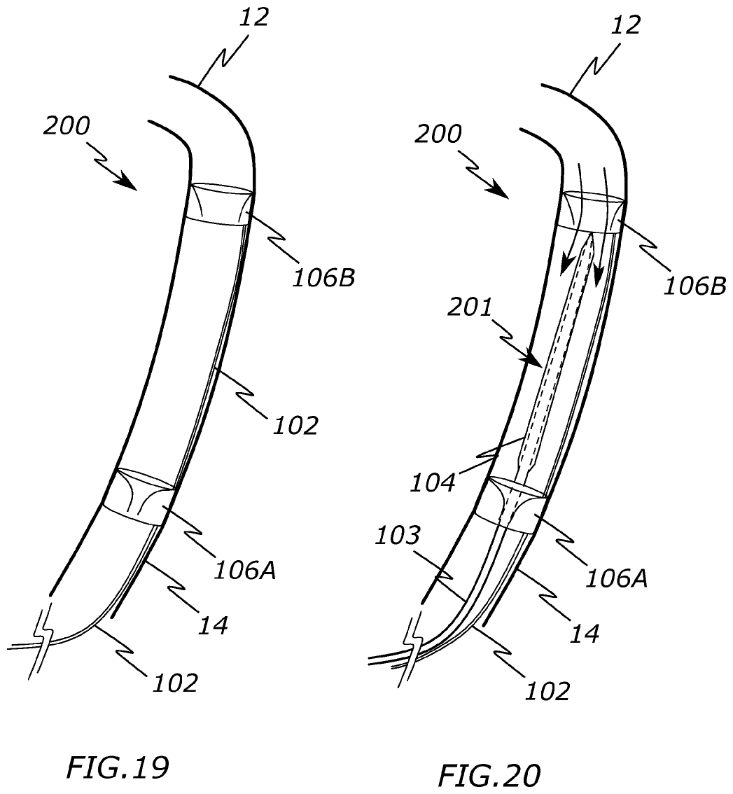

One example embodiment of this concept can be seen in FIGS. 19 and 20 which includes a valve device 200 and a separate intra-aortic balloon pump 201. The valve device 200 is shown as having a proximal valve 106A and a distal valve 106B connected on a catheter body 102. However, the valve device 200 may have any of the features or configuration described elsewhere in this specification. The intra-aortic balloon pump 201 is similar to those currently available for use today and generally include a catheter body 103 with an inflation lumen in communication with a pumping balloon 104 that is disposed on a distal portion of the catheter body 103. The valves 106A and 106B are preferably configured so that they are spaced apart sufficient to allow the pumping balloon 104 of the intra-aortic balloon pump 201 to fit. In one example, the pumping balloon 104 has a length within an inclusive range of about 22 to 27.5 cm and therefore the space between the valves 106A and 106B is at least 22 cm to 27.5 cm, though several extra centimeters may also be helpful (e.g., 25 cm to cm).

In one example, the valve device 200 is first deployed in the descending aorta 14, as seen in FIG. 19. Next, the intra-aortic balloon pump 201 is advanced through or around the proximal valve 106A so that the pumping balloon 104 is located between the two valves 106A, 106B. At that time, both devices can operate as previously discussed in this specification. Such a design may allow the physician greater choice in the type of intra-aortic balloon pump 201 that is used.

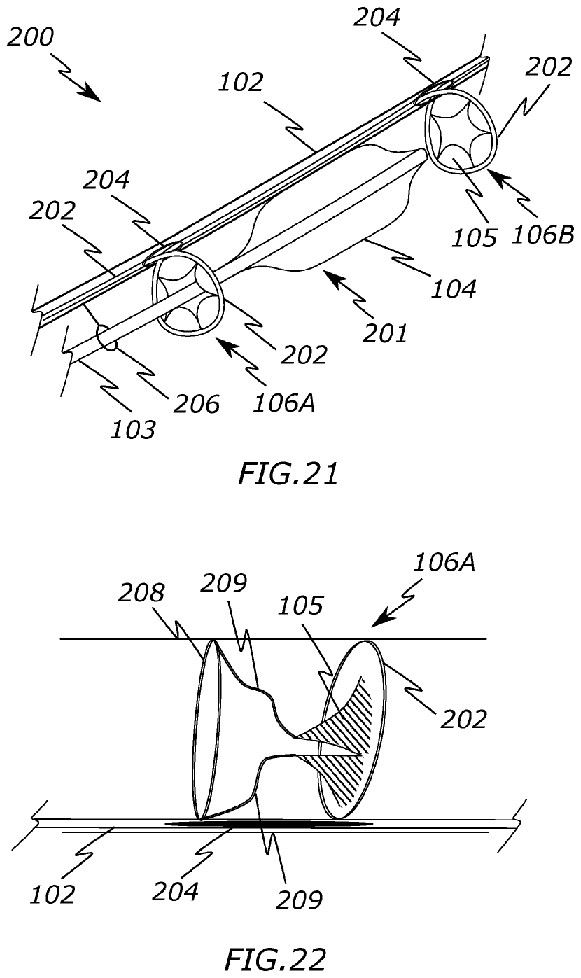

Any of the previously discussed valve types can be used for the proximal and distal valves 106A, 106B. One specific embodiment is illustrated in FIG. 21, in which the valves 106A, 106B are deployed via a control wire 202 that can be configured to either be pushed or pulled to increase the diameter and therefore deploy the valves 106A, 106B.

In one example, the control wire may extend from at or near a proximal end of the catheter body 102 and extends to a distal region of the catheter body 102. The control wire may form a loop (or alternately be attached to a loop) that forms the valves 106A, 106B. In that respect, pushing or pulling the control wire 202 may force the loop portion out of an aperture 204 in the wall of the catheter body 102. Once the loop is outside the body, it can radially expand to a circular shape onto which the valve components (e.g., valve leaflets 105) may be attached. A single control wire 202 may form the loops or connect to both valves 106A, 106B, or separate control wires 202 can be included for deployment of each valve 106A, 106B.

As seen in FIG. 21, the intra-aortic balloon pump 201 may pass through the middle of the proximal valve 106A. If that positioning is used, it may be desirable to maintain the catheter body 103 in the middle of the valve 106A. Optionally, a centering guide may be included with the valve device 202 to help maintain the catheter body 103 in the center of the valve 106A. In one example, this may include a wire 206 that extends from the catheter body 102 and engages the catheter body 103 with a complete loop or partial loop (e.g., a “C” shape). Alternately, a small balloon may be included that expands from a side of the catheter body 102 so as to contact and push the catheter body 103 to a generally center position of the valve 106A.

In some instances, using a wire loop to deploy and support a valve 106A, 106B may require additional support. In one embodiment seen in FIG. 22, additional support can be provided by providing a support scaffold 208 downstream of the valve so that it can attach to and help control and support the valve leaflets 105. In the present example, the scaffold can be a wire loop deployed similar to the previously described control wire loops with one or a plurality of tethers or arms 209 attached to both the ring and portions of the leaflets 105 (or other components of the valve 106A, 106B. These tethers or arms 209 may allow the leaflets 105 to have the appropriate range of motion to open and close, while also providing support to the valve 106A, 106B.

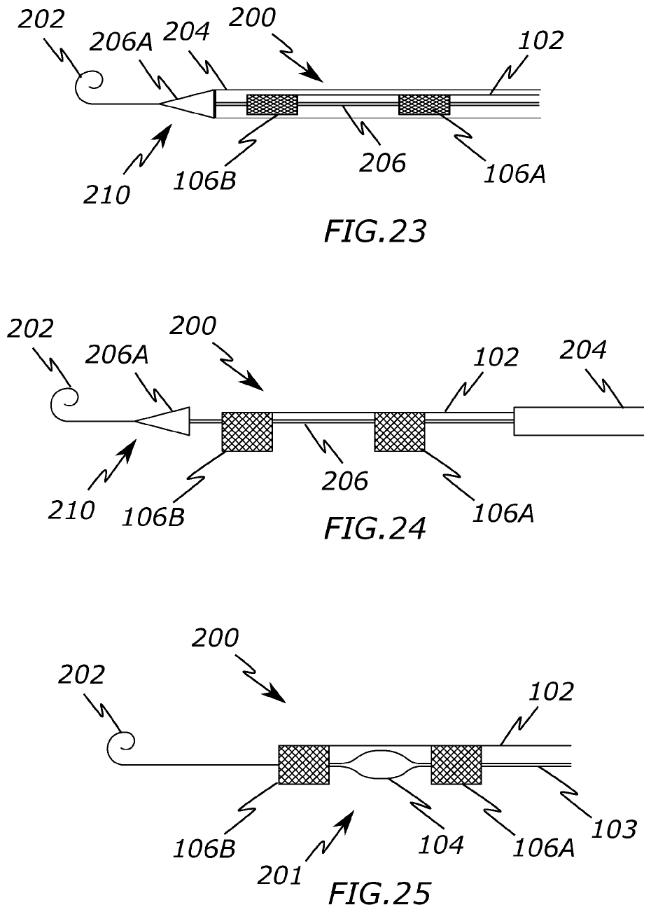

As previously discussed, the valve device 200 may either be deployed before the intra-aortic balloon pump 201 or after. FIGS. 23-25 illustrate one example of a method of deploying the valve device 200 prior to the intra-aortic balloon pump 201.

First, a guidewire 202 is advanced so that its distal end is located distally of a target deployment area (e.g., the upper end of the descending aorta 14 or even into the aortic arch 12). Next, a delivery device 210 is advanced over the guidewire so that its distal end is located near a target deployment site. In one example, the delivery device 210 includes an elongated body 206, a conical or rounded distal tip 206A positioned on the end of the elongated body 206, and an outer tubular sheath 204 that has a similar diameter as the largest portion of the tip 206A and is retractable from a proximal end of the device 210.

As seen in FIG. 23, the valve device 200 is disposed against or partially around the elongated body 206 and the outer tubular sheath 204 is initially positioned over the valve device 200. The sheath 204 maintains the radially expandable components, such as the proximal valve 106A and distal valve 106B in radially compressed positions as the delivery device 210 is advanced through the patient's vessels.

Once the delivery device 210 is in place, the sheath 204 may be retracted proximally, as seen in FIG. 24. This allows the radially expandable components, such as the proximal valve 106A and distal valve 106B, to radially expand to engage the walls of the vessel (e.g., the descending aorta 14). Once the valve device 200 is in place, the delivery device 210 is proximally withdrawn from the patient and removed from the guidewire 202.

Next, the intra-aortic balloon pump 201 is loaded onto a proximal end of the guidewire 202 and advanced distally until the balloon 104 is positioned between the two valves 106A, 106B. The valve device 200 may include a stop configured to prevent the intra-aortic balloon pump 201 from advancing too far (i.e., so that the balloon 104 does not pass into or beyond the distal valve 106B). For example, the valve device 200 may include an eyelet through which the guidewire 202 passes through but is too small for the intra-aortic balloon pump 201 to pass through. The eyelet may be positioned such that it stops movement of the intra-aortic balloon pump 201 when the balloon 104 is at a desired position between the two valves 106A, 106B. Finally, the intra-aortic balloon pump 201, as well as the valve device 200, can be operated as desired.

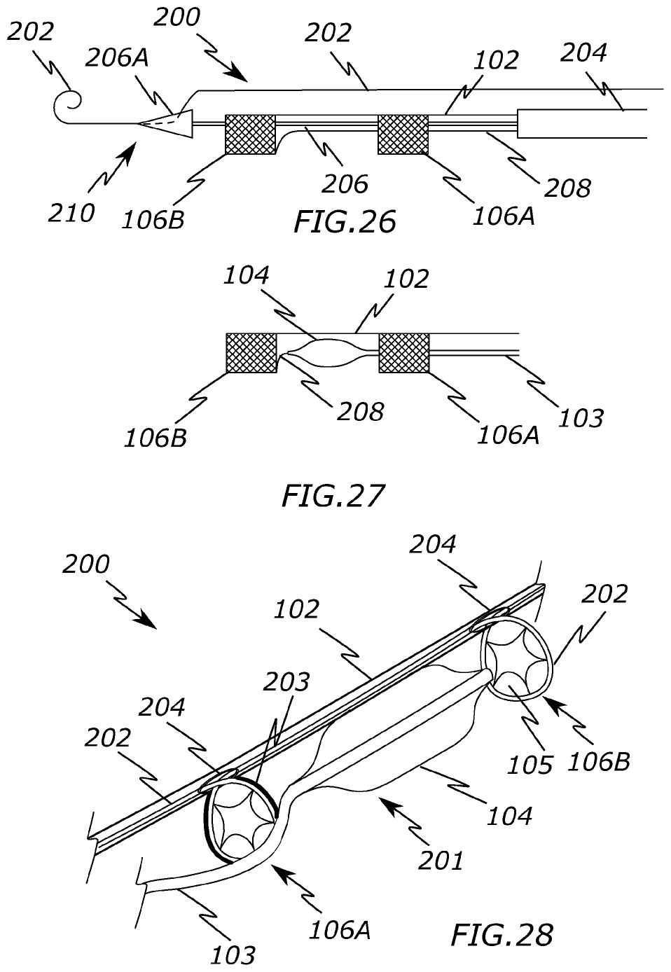

A similar approach is also possible using rapid exchange techniques. For example, the delivery device 210 may include a rapid exchange guidewire passage in its distal tip 206A, as seen in FIG. 26. Once the valve device 200 is delivered, both the guidewire 202 and the delivery device 210 may be removed. However, the valve device 200 may also include a secondary guidewire 208 that extends from its proximal end to a connection point at its distal region (e.g., near the distal valve 106B). The intra-aortic balloon pump 201 may be loaded on to the secondary guidewire 208 and advanced until it reaches the distal connection point of the secondary guidewire 208, as seen in FIG. 27. This may provide a “hard stop” that prevents advancing the intra-aortic balloon pump 201 too far distally.

As previously discussed, the catheter body 103 of the intra-aortic balloon pump 201 may alternately be positioned between the loop formed by the control wire 202 of the proximal valve 106A and the vessel wall of the descending aorta 14, which can be seen in FIG. 28. This arrangement may be particularly helpful if the physician delivers the intra-aortic balloon pump 201 first, and the valve device 200 second. In such arrangements, it may be helpful to include a sealing member 203 to seal between the outer surface of the ring of the proximal valve 106A and the catheter body 103 of the intra-aortic balloon pump 201. The sealing member can be fixed to the outer circumference of the loop of the proximal valve 106A, as seen in FIG. 28, or can alternately be fixed along a length of the catheter body 103 that is aligned with the proximal valve 106A.

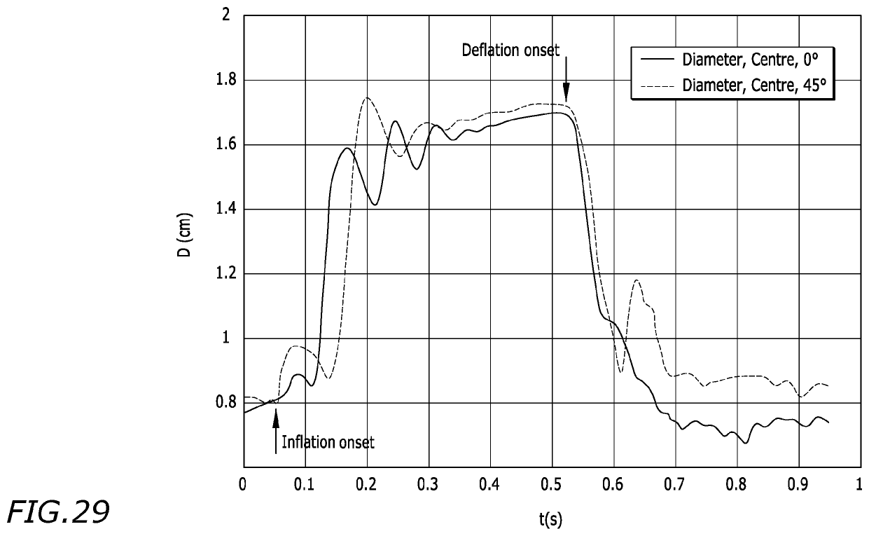

One apparent limitation to the speed an intra-aortic balloon pump may pump blood is the speed at which the balloon can deflate. This can be seen in FIG. 29 in which deflation can take several milliseconds. In that respect, any of the embodiments of this specification may include pumping balloons with features that enhance deflation. Generally, these deflation features may mechanically apply force to the pumping balloon so that it deflates faster.

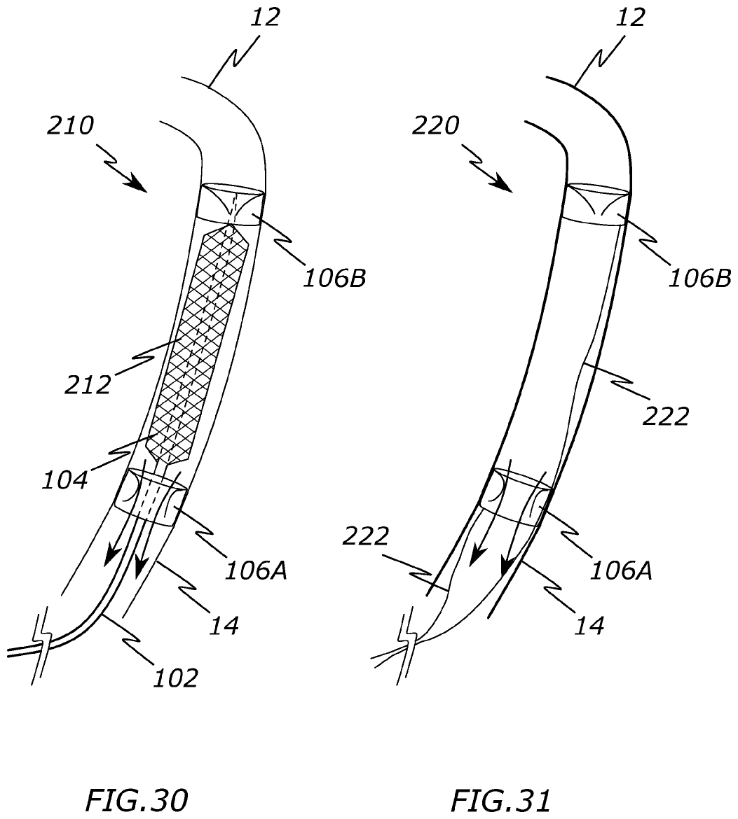

In one embodiment, FIG. 30 illustrates an intra-aortic balloon pump 210 that includes a framework 212 around its pumping balloon 104. In one example, the framework 212 can be a braided or laser-cut cage surrounding the pumping balloon 212. The cage may be composed of a super elastic alloy, such as Nitinol, that has a heat set shape in a radially compressed configuration but can also expand to a radially expanded configuration as the balloon 104 pushes outwards. Hence, the cage can apply radially compressive force that accelerates deflation.

In another example, an elastic tube or elastic bands may be placed around the pumping balloon 104 to achieve a similar radially compressive force that accelerates deflation.

Generally, there may be many different approaches to the timing of the inflation/deflation pump cycle during a cardiac cycle. For example, a pump cycle may occur one or more times per cardiac cycle (e.g., 1, 2, 3, 4 times). In another example, the pump cycle may occur less than one time per cardiac cycle, such as every other cardiac cycle or every third cardiac cycle. Operating the pump cycle less than once per cardiac cycle may be beneficial in limiting the amount of blood pulled from the arteries in the aortic arch, such as the cerebral and coronary arteries.

In another specific example, the intra-aortic balloon pump may also perform some cycles with both its proximal and distal valves open. For example, during a first cardiac cycle, the two pump cycles may occur with the valves in normal operation, and then in a second subsequent pump cycle, the pumping balloon may pump twice with both valves remaining in a wide open state (e.g., with balloon valves). This may allow for a cardiac cycle with high efficiency pumping and a second cardiac cycle with less efficiency that may reduce blood pull from arteries in the aortic arch, such as the cerebral and coronary arteries.

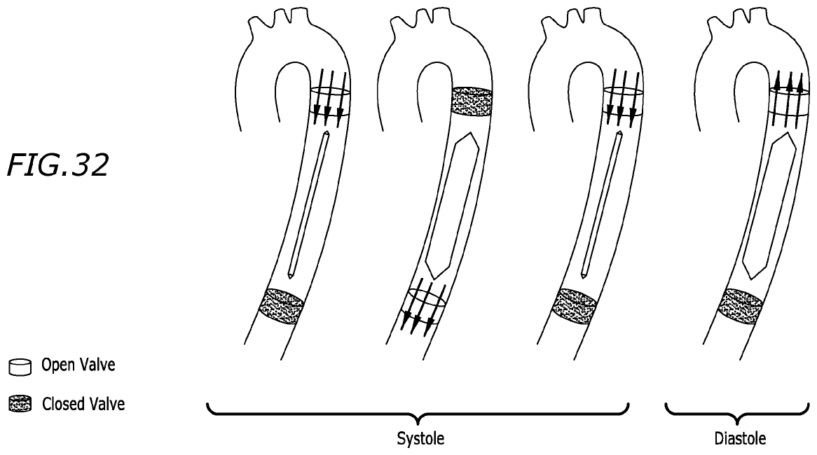

In another specific example, the intra-aortic balloon pump may also be used to pump blood retrograde during some cardiac cycles. For example, as seen in FIG. 32, a normal pump cycle can be performed during systole that pushes blood in an antegrade direction. However, during diastole, the proximal valve may be closed and the distal valve may be opened while the pumping balloon is inflated so as to push some blood in a retrograde direction. Again, this may push blood into arteries in the aortic arch, such as the cerebral and coronary arteries.

While the proximal and distal valves 106A, 106B have been described as being attached to a catheter body 102, in some embodiments the valves 106A, 106B may have a self-expanding stent-like outer body that are each separately deployed and anchored alone without any permanent connection to a catheter body 102. This may allow an intra-aortic balloon pump 201 having a pumping balloon 104 and no proximal/distal valves to be positioned and used between the individually deployed valves 106A, 106B.

These individually deployed and anchored valves 106A, 106B may also include a mechanism for retrieval from the patient later. In one example seen in FIG. 31, such a retrieval system may include a retrieval tether 222 connected to the valves 106A, 106B. Either each valve 106A, 106B may include its own tether 222 or both valves may be connected to the same tether 222. The tether(s) 222 may extend outside the patient and allow a retrieval device to be advanced over the tether 222 later for removal. Optionally, the tether 222 may be connected to the valves 106A, 106B in a way that applying tension reduces the diameter of the valves 106A, 106B.

While the embodiments of the intra-aortic pumping balloon of this specification are primarily discussed and shown in the context of use in the descending aorta 14, as well as the aortic arch 12 and ascending aorta 18, any of these embodiments can be used at different locations for similar or different uses. For example, any of these pumping balloon embodiments can be used in or near the abdominal or thoracic aorta, on the venous side by the renal arteries to pull blood flow, in the superior vena cava in combination with a second double valve arterial intra-aortic balloon pump to help increase cerebral gradient, between the superior vena cava and the inferior vena cava in combination with an arterial double valve intra-aortic balloon pump to adjust capillary gradient, in the ascending aorta, in the aortic arch, in the subclavian artery, in the carotid artery.

The following embodiments are generally directed to devices that are positioned within a patient's heart to increase the amount of blood pumped. These devices may be used independently of the previously described intra-aortic balloon pump embodiments or may be used with any of those embodiments.

A ventricular assist device (VAD), also known as a mechanical circulatory support device, is an implantable mechanical pump that helps pump blood from the ventricles of a patient's heart to the rest of the body. A ventricular assist device is used in people who have weakened hearts or heart failure.

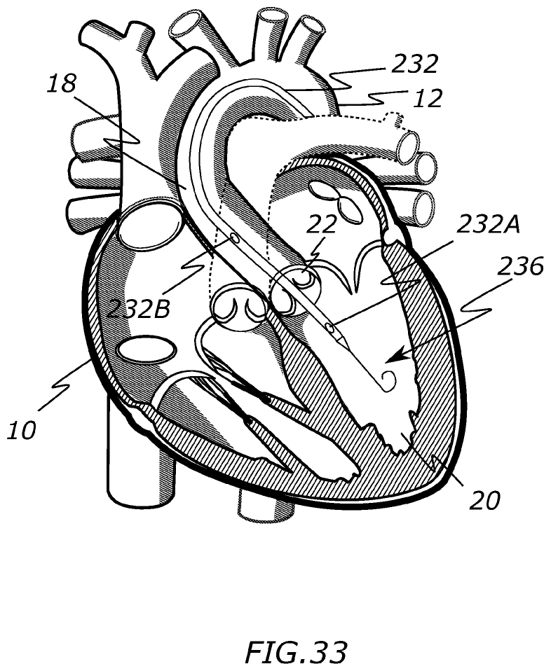

One popular type of ventricular assist device is known as an Impella device 236, shown in FIG. 33, which is a miniaturized ventricular assist device that includes a catheter body 232, an inlet 232A into an internal passage of the catheter body 232, an impeller within the internal passage, and an outlet 232B. The inlet 232A is typically located near the distal end of the catheter body 232, while the outlet 232B is typically located several centimeters proximally.

Although an Impella device can be placed in the left, right or both ventricles of your heart, it is most frequently used in the left ventricle 20, as seen in FIG. 33. Typically, the inlet 232A is positioned in the left ventricle 20, while the outlet 232B is positioned on the other side of the aortic valve 22, within the ascending aorta 18 or aortic arch 12. When the impeller within the internal passage is activated, it draws in blood from the left ventricle 20 and passes it into the ascending aorta 18 or aortic arch 12, thereby increasing blood flow into the aorta and assisting the patient's heart 10.

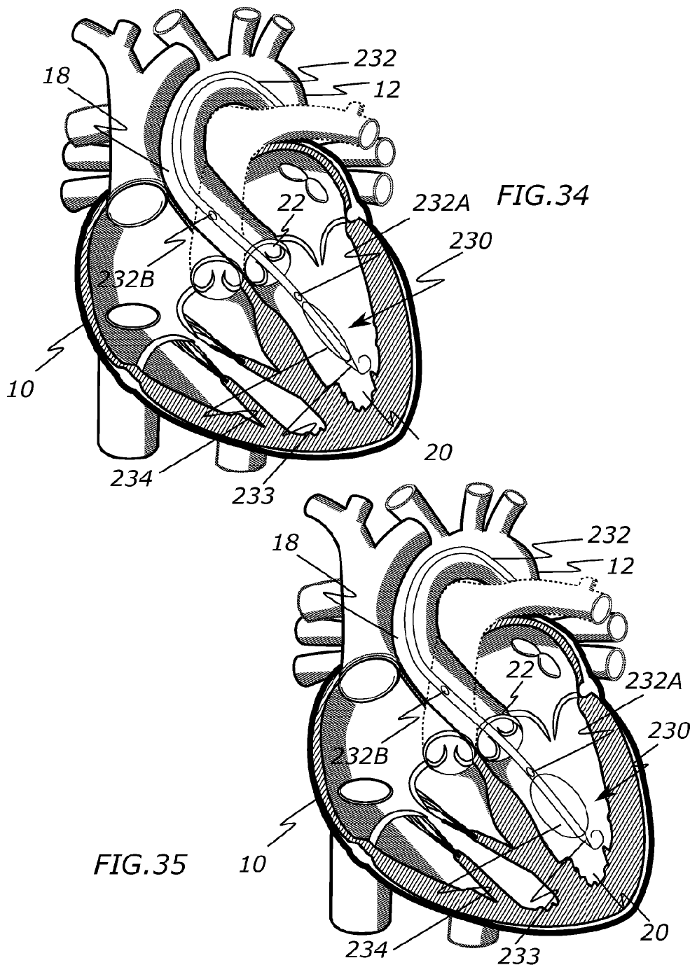

Referring to FIGS. 34 and 35, a modified ventricular assist device 230 is illustrated which may be similar to the previously described Impella device 236, but further includes a counter pulsation balloon 234 located at a distal end of the catheter body 232 and configured to inflate and deflate during a cardiac cycle to further improve blood pumping. Specifically, the counter pulsation balloon 234 may be configured to inflate during diastole and deflate during systole.

The counter pulsation balloon 234 may be located distally of the outlet 232A or proximally, as long as it does not block or otherwise cause the outlet 232A to be obstructed during use. The catheter body 232 may include an inflation lumen that connects at a distal end of the catheter body 232 to tubes that are connected to a control device (similar to the previously described IABP control device 110). The control device 110 may also be connected to sensors on the patient (e.g., ECG and/or blood pressure) to determine the desired inflation and deflation time for the counter pulsation balloon 234.

One goal of the counter pulsation balloon 234 is to reduce pressure in the left ventricle 20 in the presence of the pumping of the Impella/VAD pumping features to allow for myocardial recovery in acute cases and/or ventricular remodeling in chronic cases.

In one example, the counter pulsation balloon 234 inflates slowly during relaxation of the left ventricle 20 (diastole, FIG. 35) and deflates rapidly during contraction of the left ventricle 20 (systole, FIG. 34). This allows the Impella/VAD pumping features to maintain about 5 L/min of circulatory support while the balloon reduces pressure in the left ventricle 20, which thereby may reduce the work of the left ventricle 20, may reduce myocardial oxygen demand, and may improve the coronary flow.

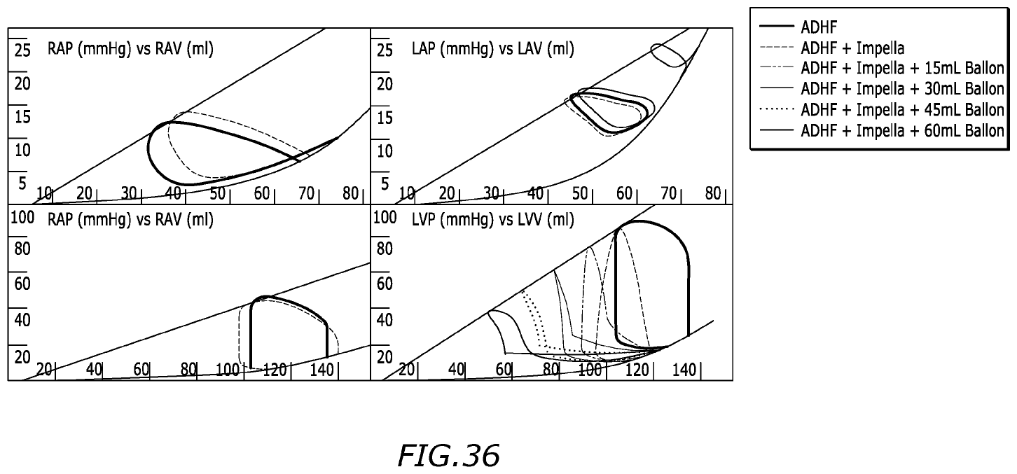

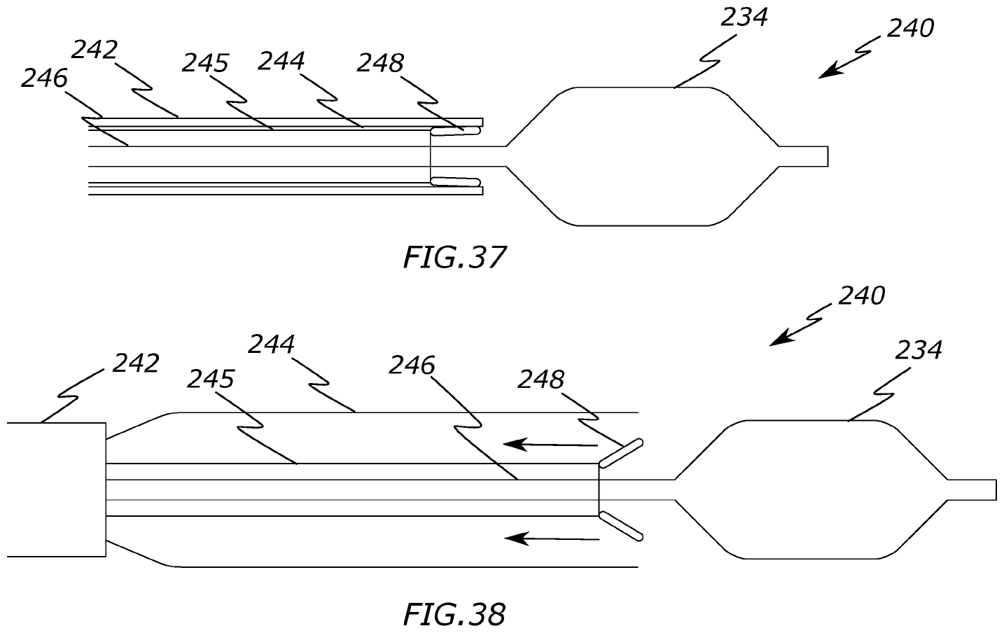

FIG. 36 illustrates some example results showing that left ventricle pressures decrease linearly with balloon volume and left atrium pressures do not increase significantly. Note, ADHF indicates acute decompensated heart failure in a patient's heart, “Impella” indicates the use of Impella/VAD pumping features, and the various volumes indicate the volume of the counter pulsation balloon 234. Similarly, FIGS. 37-40 illustrate how the effect of the counter pulsation balloon 234 scales linearly with balloon volume. Left ventricle pressure volume (PVA), which is a measure of the work the ventricle is performing, decreases dramatically (e.g., about 50% reduction with a balloon. Left ventricle myocardial oxygen consumption (LV MVO2) decreases, which may reduce ischemic damage over time. Also, the left ventricle end diastolic pressure (LVEDP) shows only minimal increase, while the left ventricle end systolic pressure (LVESP), i.e., the afterload, shows a large decrease.

The counter pulsation balloon 234 may have a variety of different volume sizes, including 15 ml, 30 ml, 45 ml, and 60 ml of volume.

The ventricular assist device 230 may also include a sensor 233 to sense various characteristics in the patient. The sensor 233 is illustrated in the distal tip of the catheter body 232, however, it can be located at various locations along the length of the catheter body 232. Additionally, one or more sensors 233 can be included.

In one example, the sensor 233 is a temperature sensor that is configured to sense temperature within the left ventricle 20. Increased temperature values may indicate that the left ventricle 20 is performing increasing work, while decreased temperature values may indicate that the left ventricle 20 is performing a decreased amount of work.

In another example, the sensor 233 is a myocardial pressure sensor that is configured to improve the timing of the inflation of the counter pulsation balloon 234 by sensing the contraction of the heart 10. Additionally, the sensor may be an EKG sensor for sensing local changes in electrical activation.

While the embodiment of the ventricular assist device 230 of FIGS. 34 and 35 is depicted as having an aperture for the inlet 232A, other inlet and outlet structures are also possible. For example, FIGS. 37 and 38 illustrate a ventricular assist device 240 with a radially expandable inlet. More specifically, a radially expandable sleeve 244 may be expanded positioned at a distal portion of the device 230 and can be selectively expandable during use. This allows the device 230 to maintain a relatively small diameter profile during advancement and placement but provides a relatively larger inlet opening to pump blood.

The device 240 includes an inner elongated body member 246 which is connected to the counter pulsation balloon 234 on its distal end. The body member 246 includes an inflation passage connected to the balloon to allow inflation of the balloon 234. A second tubular body member 245 may be disposed around the inner elongated body member 246 and supports an impeller 248 at or near its distal end. In some embodiments, the impeller 248 is composed of a flexible material that allows it to be radially compressed and radially expanded. For example, the impeller can be composed of Nitinol and shape set to a desired impeller shape when unconstrained. The second tubular body member 245 also contains the necessary components to cause the impeller 248 to rotate, such as a motor and electrical circuit.

The radially expandable sleeve 244 is disposed around the second tubular body member 245 and the impeller 248 and forms a passage that connects or is in communication with an outlet (e.g., 232B) at a proximal location of the aortic valve 22. the sleeve 244 can be radially expanded and contracted in various ways, including by use of an outer, slidable sheath 242. When the sheath 242 is positioned over the sleeve 244, it maintains the sleeve 244 in a radially compressed configuration, substantially closing off the pumping passage and optionally compressing the impeller 248. When the outer sheath 242 is proximally retracted, it exposes the sleeve 244, allowing it and optionally the impeller 248 to radially expand, opening up the pumping passage. Other mechanisms for expanding the sleeve 244 are also possible, such as a pull wire mechanism.

The sleeve 244 may be composed of a blood impermeable material that is biased to radially expand when unconstrained. For example, the sleeve 244 may be composed of a braided or laser cut stent-like structure with a polymer, blood impermeable layer disposed over or under it.

While not shown in these figures, the proximal outlet may also include a feature that maintains it in a closed configuration during placement and then can be opened after delivery. For example, the proximal outlet of the pumping passage may extend through an aperture of the outer sheath 242 if the sheath 242 extends back to the proximal end of the device 240. This aperture in the outer sheath 242 may be sized and positioned such that it does not connect with the underlying pumping passage created with the sleeve 244 when the sheath 242 is in its distal position. However, when the sheath 242 is retracted to its proximal position, its aperture aligns with an opening to the pumping passage, thereby creating a pumping passage continuously through the device 240.

In any of the ventricular assist device embodiments, the counter pulsation balloon 232 may be filled with a gas. The inflation/deflation cycles may be active (e.g., a gas such as helium is quickly injected and removed from the balloon). Alternately, the discharge cycle may have a passive component to it. Specifically, a scaffold or band that provides constant radially compressive force (similar to that previously described in other embodiments) so that the discharge time can be decreased or even performed passively without any electrical pump assistance.

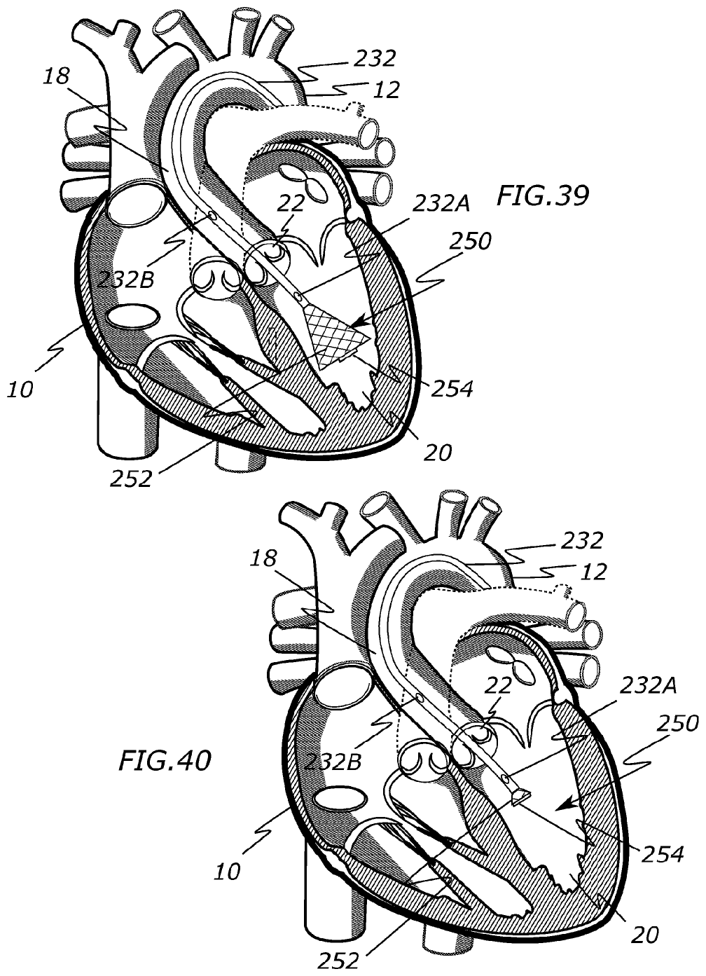

In addition to a balloon, other mechanisms may alternately be used for a counter pulsation balloon within the left ventricle 20. For example, FIGS. 39 and 40 illustrate a ventricular assist device 250 that is generally similar to the previously described device 230, however, instead of a distal counter pulsation balloon 234, an expandable structure 254 can be distally pushed out of the catheter body 232 and retracted, similar to rapidly expanding and deflating a balloon.

The expandable structure 254 may have a somewhat rigid scaffold structure that is configure to radially expand as it is pushed out of the catheter body 232. For example, the scaffold structure may be composed of a braided mesh or laser-cut struts. Nitinol or a similar shape memory material may also be used for the scaffold such that it expands to its larger, expanded shape when outside of the catheter body 232. The scaffold structure is preferably covered on its outside or inside such that it creates a blood impenetrable barrier that displaces blood as it expands. Optionally, the expandable structure 254 may have a one-way valve that prevents blood from entering the expandable structure 254 but allows fluid inside to quickly escape and thereby allowing the expandable structure 254 to quickly radially compress and contract back into the catheter body 232 in a manner similarly described for counter pulsation balloon 234.

The expandable structure 254 may have a variety of different expanded shapes. For example, it may have a conical shape, a rounded shape, and oval shape, or other similar shapes.

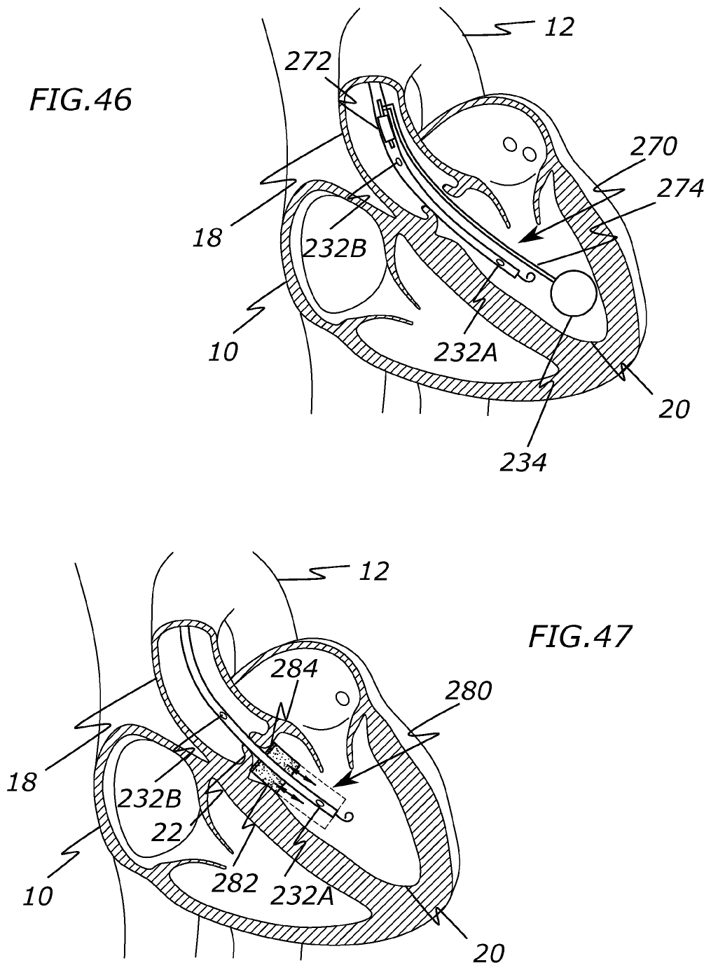

FIGS. 45 and 46 illustrate a similar embodiment of a ventricular assist device 260 that is similar to the previously described device 250, including having an expandable structure. However, the device 260 has a somewhat different scaffold structure.

Specifically, its scaffold comprises an elongated control wire 264 that extends to a proximal end of the device to allow the control wire 264 to move proximally and distally relative to the outer catheter body 232. The ends of a second wire 262 are connected at or near the distal tip of the control wire 264 and to the catheter body 232. The second wire 262 is also helically wound around the control wire 264.

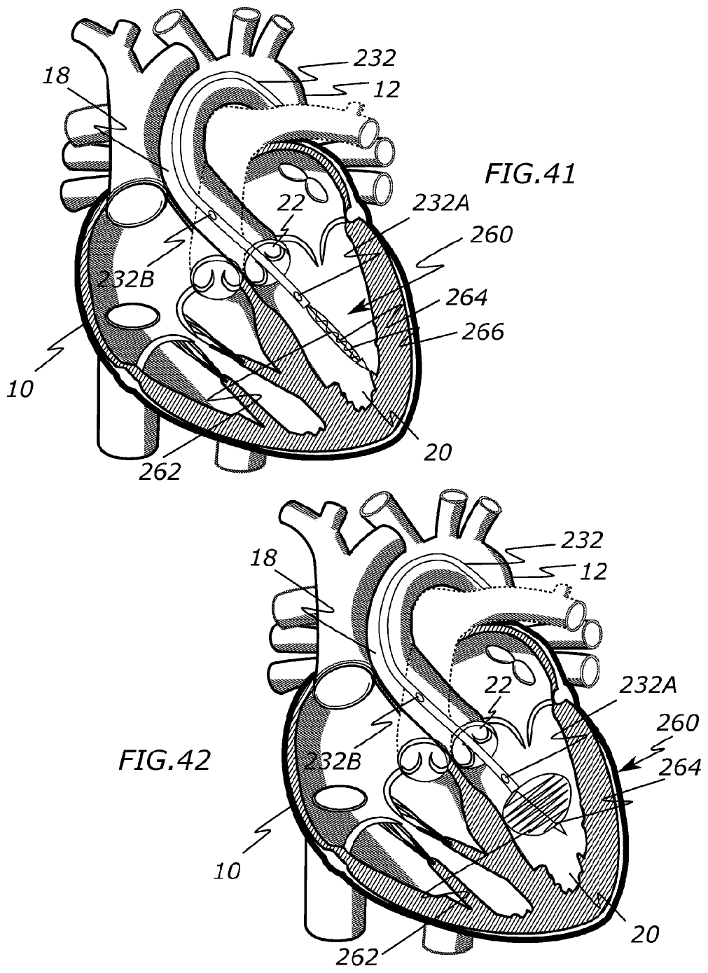

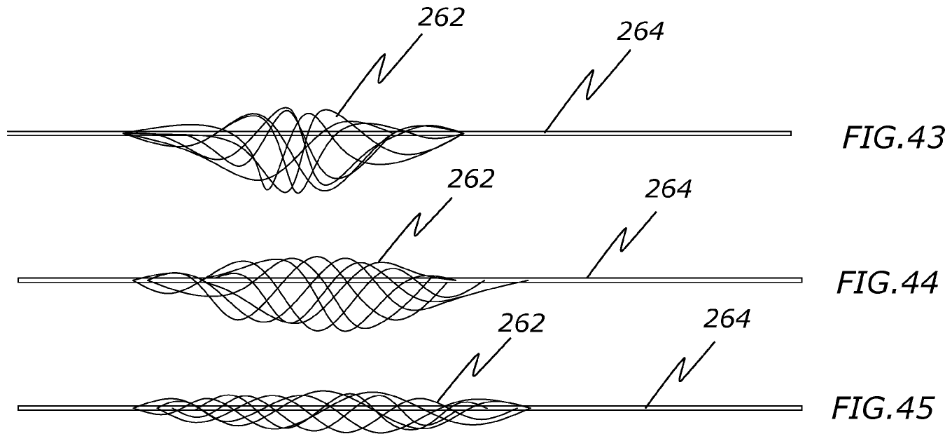

When the control wire 264 is extended distally out of the catheter body 232, the helically wound second wire 262 remains radially compressed and close to the diameter of the control wire 264, as seen in FIG. 41. When the control wire 264 is proximally retracted, the helically wound second wire 262 radially expands outwards, as seen in FIG. 42. A blood impenetrable cover or membrane 266 is disposed over the second wire 264, so that when the second wire 262 radially expands, the cover 266 displaces blood. Hence, proximal and distal movement of the control wire 264 can expand and contract the membrane, acting similar to the previously described counter pulsation balloon.

While the second wire 262 is described as a single helically wound wire, a plurality of wires may also be used. For example, FIGS. 47-49 illustrate a control wire 264 with a plurality of helically wound wires 262. These wires 262 may be only helically wound or may be interwoven or braided with each other to form a braided mesh scaffold.

In some embodiments, the impeller mechanism may help with the inflation of the counter pulsation balloon 234 of a ventricular assist device. For example, FIG. 46 illustrates a generally similar embodiment of a ventricular assist device 270 that is similar to the previously described device 230, except that it has a biased, releasable pressure reservoir 272 in which blood builds up and then can be released into balloon tube 274 to fill the counter pulsation balloon 234.

The pumping passage between the inlet 232A and outlet 232B may include a further passage that it is in communication with. This further passage diverts some of the pumped blood into the reservoir 272, allowing pressure to build up. The pressure reservoir may include a pneumatic or hydraulic cylinder and have an internal bias or energy storage means. The reservoir may also include a first valve between it and the pumping passage, as well as a second valve between it and the passage to the counter pulsation balloon 234.

During diastole, the first valve opens to divert blood into the pressure reservoir 272 to reduce the volume of the pressure reservoir 272 to increase the volume and pressure in the counter pulsation balloon 234. At the end of diastole, the first valve closes to stop further blood diversion into the reservoir 272. During systole, the pressure against the pressure reservoir 272 decreases which causes the balloon 234 to at least partially collapse and expand the pressure reservoir 272. The re-expansion of the pressure reservoir 272 may be aided by an internal bias mechanism (e.g., a spring). At the end of systole, the first valve opens and the second valve closes to initiate the next cycle.

In some embodiments, a counter pulsation balloon can be configured to expand in length. For example, FIG. 47 illustrates ventricular assist device 280 that is generally similar to the previously described device 230. However, the device 280 includes a counter pulsation balloon 282 that increases and decreases in length, allowing the balloon 282 to expand downward from are area adjacent to the aortic valve 22.

The device 280 may include a collar 284 that provides a rigid backstop against which the balloon 282 expands from. The collar 284 may be composed of an expandable metal framework (e.g., braided or laser-cut Nitinol) or may be an inflatable balloon. The collar 282 may also include a sealing material on its outer surface to help seal against the aortic valve 22 and/or the aortic annulus to prevent blood flow around the collar 284. Additionally, the collar may include one or more anchors (e.g., barbs, spikes, etc.) to help anchor the collar 284 in place.

The counter pulsation balloon 282 may be attached to the collar 284 in a manner that allows it to generally expand in a desired direction. For example, the balloon 282 may be configured to expand distally so that, when placed the left ventricle 20, it expands away from the aortic valve 22 and deeper into the chamber of the left ventricle 20.

The counter pulsation balloon 282 may be composed of an anisotropic material that allows the balloon 282 to expand in one direction and limits expansion in a second direction. Alternately, the balloon may include material that restricts side expansion, such as a noncompliant polymer band or a braided mesh band. The counter pulsation balloon 282 may have a single chamber or a plurality of chambers. The material of the balloon 282 may be comprised of a silicone or polyurethane material, and may be heat-deformed.

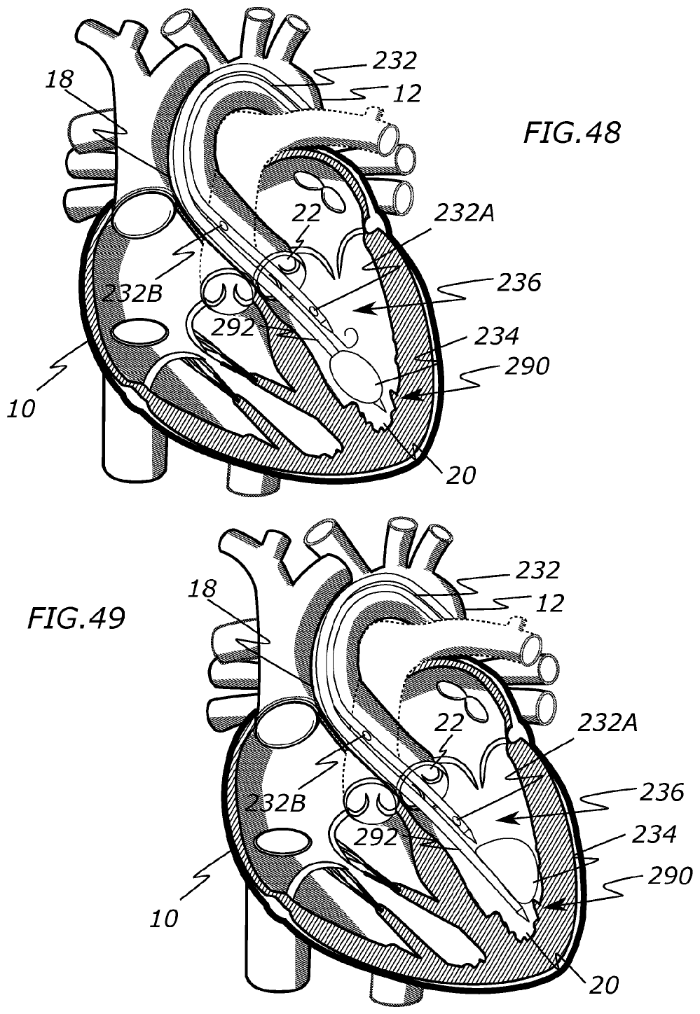

While various embodiments have described the counter pulsation balloon 234 as being located on the same catheter as the ventricular assist device, in other embodiments, the counter pulsation balloon 234 can be located on a second, separate catheter. For example, FIG. 48 illustrates a counter pulsation balloon catheter 290 having a catheter body 292 with an inflation passage extending between a proximal and distal region of the catheter body 292. The counter pulsation balloon 234 is fixed at the distal region of the catheter body 292 and is in communication with the inflation passage, allowing the counter pulsation balloon 234 to inflate and deflate as necessary while the ventricular assist device 236 operates as previously described. The counter pulsation balloon catheter 290 can be delivered to the left ventricle 20 prior to the ventricular assist device 236 or after.

The counter pulsation balloon 236 can be configured to expand in a generally symmetrical manner as seen in FIG. 48 or can be configured to expand asymmetrically relative to an axis of the catheter body 292 (e.g., from one side of the catheter body 292), as seen in FIG. 49.

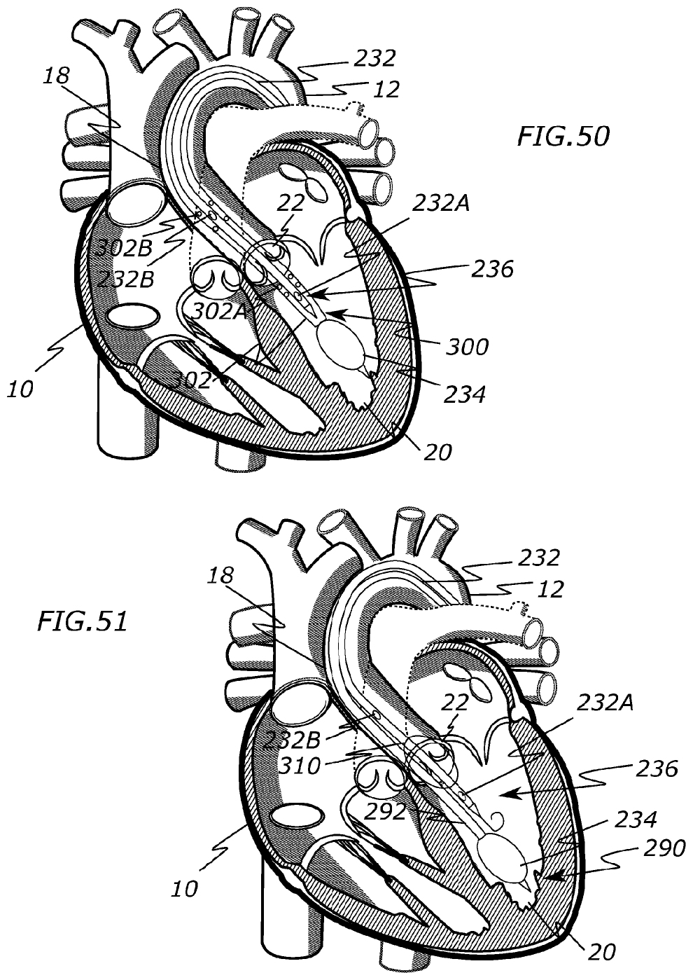

In another embodiment, the counter pulsation balloon 234 may be located on a larger catheter or sheath through which the ventricular assist device 236 may pass through. For example, FIG. 50 illustrates a counter pulsation catheter 300 having an elongated catheter body 302 with both an inflation lumen for the counter pulsation balloon 234 fixed at its distal end, and a second passage sized for the ventricular assist device 236. In this respect, an existing, “off-the-shelf” ventricular assist device 236 can be used with the catheter 300.

The counter pulsation catheter 300 may further include openings or apertures that open into the ventricular assist device passage of the catheter body 302, allowing blood to enter the passage via distal apertures 302A, enter inlet 232A of the ventricular assist device 236, be pumped out the outlet 232B, and then pass through proximal apertures 302B. Optionally, the ventricular assist device passage may include one or more seals or barriers that extend against the ventricular assist device 236 to help create a channel between the apertures 302A, 302B and the inlet and outlet 232A, 232B.

It may be desirable to occlude the aortic valve 22 during a procedure to prevent regurgitation. Hence, any embodiments of this specification may further include a valve occlusion device. In one embodiment, this aortic valve closure can be achieved with an occlusion balloon 310, as seen in FIG. 51. The occlusion balloon 310 may be connected to and expand from either the counter pulsation catheter 290, the ventricular assist device 236, or embodiments in which both the ventricular assist components and counter pulsation balloon 234 are included in one device. The occlusion balloon 310 is connected to an inflation passage and is sized, in its inflated state, to occlude an aortic valve 22.

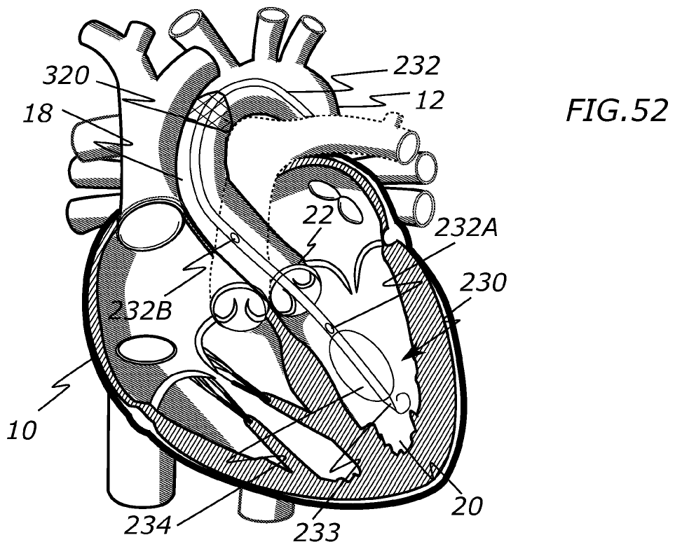

Any of the embodiments of this specification may include an embolic protection device that is configured to capture embolic material. For example, FIG. 52 illustrates a vascular assist device 230 that includes an embolic protection device 320. The embolic protection device 320 expand from a compressed configuration to an expanded configuration that is generally sized to engage a wall of the surrounding vessel. The embolic protection device 320 can be a filter, such as braided mesh with a pore size sufficient to allow passage of blood but prevent passage of most embolic material. The embolic protection device 320 may be positioned at a variety of different locations proximal of the distal end of the device. In the present embodiment, the embolic protection device 320 is positioned proximally of the outlet 232B and within the ascending aorta 18 or aortic arch 12.

Any of the counter pulsation balloon catheters described in this specification can be used with any of the intra-aortic balloon catheters described in this specification. Additionally, the features of any of these two catheters may be combined into a single catheter.

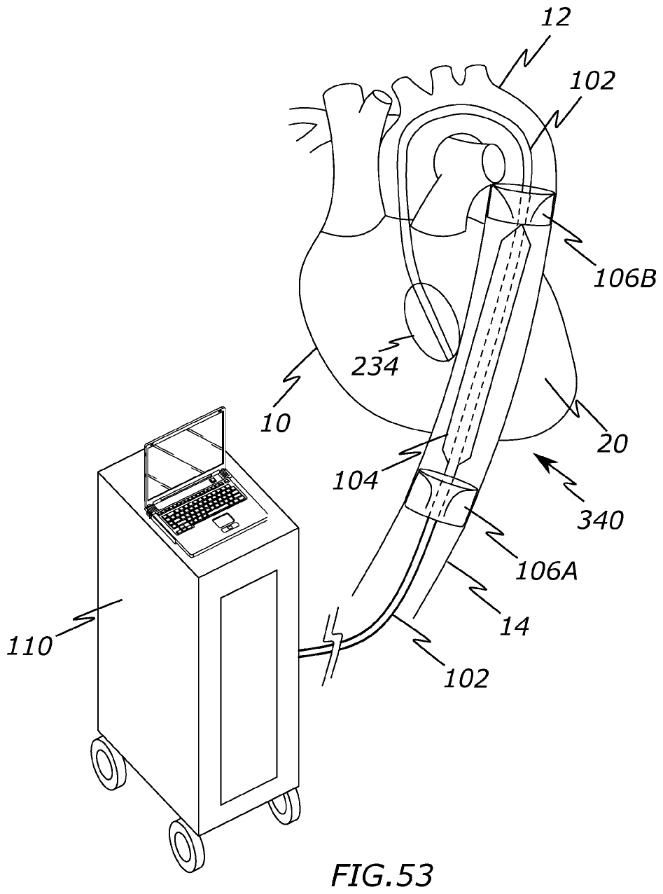

For example, FIG. 53 illustrates a catheter 340 that includes both a pumping balloon 104 configured for positioning within the descending aorta 14 and a counter pulsation balloon 234 configured for placement within the left ventricle 20. Both the balloons 104, 234 are similar to those previously described and used in a similar manner (e.g., both inflated during diastole and deflated during systole). Optionally, the proximal valve 106A and distal valve 106B may be included but may not be necessary. Additionally, the catheter 340 does not include any ventricular assist device components (e.g., pumps, impellers, etc.) since both balloons 104, 234 may provide enough assistance, however other embodiments may include the previously described ventricular assist components.

Generally, the balloons 104, 234 are configured to inflate at about the same time. For example, the balloons 104, 234 may both inflate during diastole and deflate during systole. Each balloon 104, 234 may have their own separate inflation lumens or they may be connected to the same inflation lumen. In either case, the catheter 340 is configured such that both balloons 104, 234 inflate at about the same time. Since the counter pulsation balloon 234 is positioned at the distal portion of the catheter 340, in a further distal location relative to the pumping balloon 104, and in a location (the left ventricle 20) which may have slightly different pressure within, there may be more or less resistance to inflating the counter pulsation balloon 234. Hence, it may be desirable to include a structure to adjust the inflation lumen or the opening to each balloon 104, 234 to account for any resistance or differences between the balloons. In other words, one balloon 104, 234 may experience greater pressure than the other to achieve more uniform inflation.

For example, each balloon 104, 234 may have separate inflation lumens and separate connections to a control device 110 that can independently cause them both to inflate at the same time.

In another example, catheter 340 may have a single inflation lumen connected to a single inflation tube/connection to the control device 110. The opening to the pumping balloon 104 from the inflation lumen may be somewhat smaller than the opening to the counter pulsation balloon 234, allowing both to inflate at about the same time and same speed (i.e., it has a region of narrowed diameter smaller than any portion connected to the other balloon).

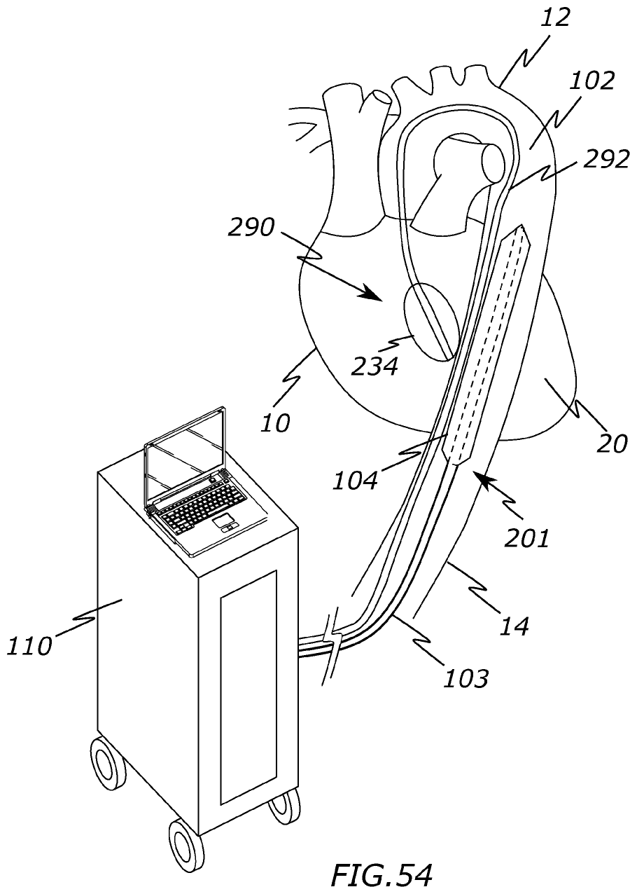

In another example, FIG. 54 illustrates a counter pulsation balloon catheter 290 used with a separate intra-aortic balloon pump 201, both of which have been previously described in this specification. Again, the balloons 104, 134 can be positioned and used as previously discussed. However, since the catheters 201, 290 are separate, they may be separately placed (e.g., the counter pulsation balloon catheter 290 may be placed in the left ventricle 20 first and then then the intra-aortic balloon pump catheter 201 may be placed within the descending aorta 14).

While both catheters 201, 290 may have separate inflation lumens/tubes that directly connect to a control device 110, it may also be desirable to use an existing “off the shelf” control device designed solely for existing intra-aortic balloon pump catheters 201. In that respect, the control device 110 may only have one inflation lumen and an adapter may be necessary to split the inflation lumen between the two catheters 201, 290. In such cases, it may be desirable to adjust or restrict inflation to one of the catheters so that both balloons 104, 234 inflate and deflate at the same time. Hence, any such adapter may include an inflation adjustment mechanism. This inflation adjustment mechanism may allow gas or fluid to enter balloon 104 in a somewhat slower manner so that it can match the inflation time/rate of balloon 234 which may have additional resistance on inflation due to the longer catheter length and position within the heart 10 or from other factors.

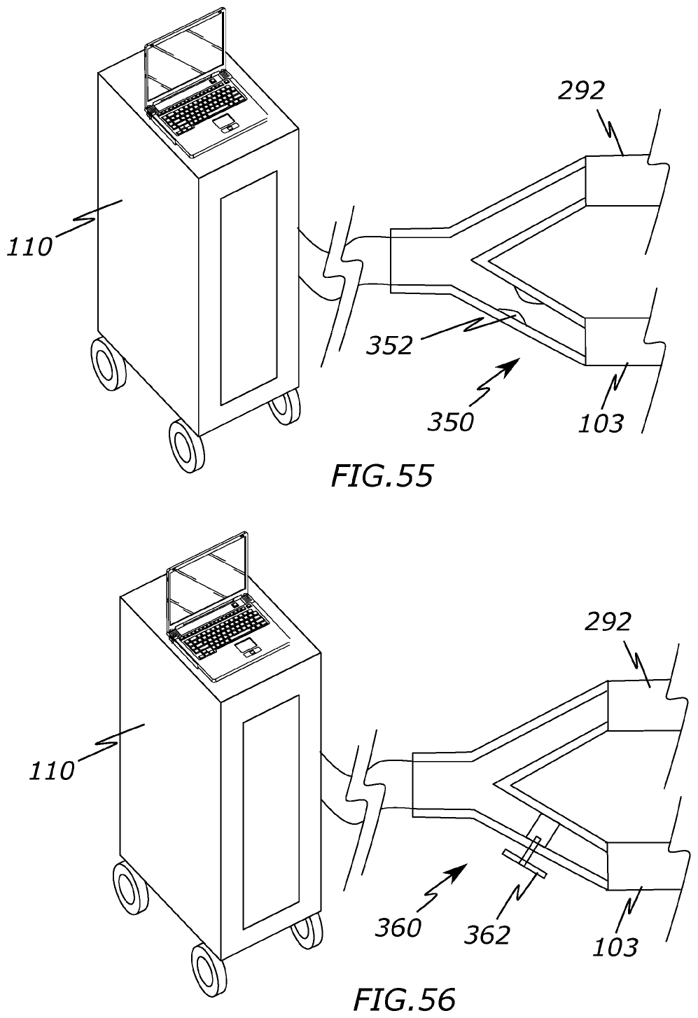

For example, FIG. 55 illustrates a Y adapter 350 that has an inflation lumen that is configured to connect to an inflation control device 110, as well as the catheter body 292 of the counter pulsation balloon catheter 290 and a catheter body 103 of the intra-aortic balloon pump 201. The Y adapter 350 may include an inflation adjustment mechanism comprising a region 352 of the inflation lumen of the adapter 350 having a reduced diameter leading to the catheter body 103. This may decrease the rate of inflation of the pumping balloon 104 relative to the counter pulsation balloon 234 so that they inflate at about the same time and/or rate.

Depending on which type and/or size of the intra-aortic balloon pump 201, it may be desirable for the physician to adjust the inflation rate of one of the catheters inflation lumens relative to the other so that they can fully inflate at about the same time (i.e., inflating during diastole and deflating during systole). For example, FIG. 56 illustrates a Y adapter 360 that has an inflation lumen that is configured to connect to an inflation control device 110, as well as the catheter body 292 of the counter pulsation balloon catheter 290 and a catheter body 103 of the intra-aortic balloon pump 201. However, the adapter 360 includes an adjustment mechanism 362 that allows a user to increase or decrease the amount of media (gas or fluid) through the passage and to allow the passage of the adapter 360 to inflate both balloons 201, 234. The adjustment mechanism 362 may include an external knob that moves into or out of the inflation passage to increase or decrease its size.

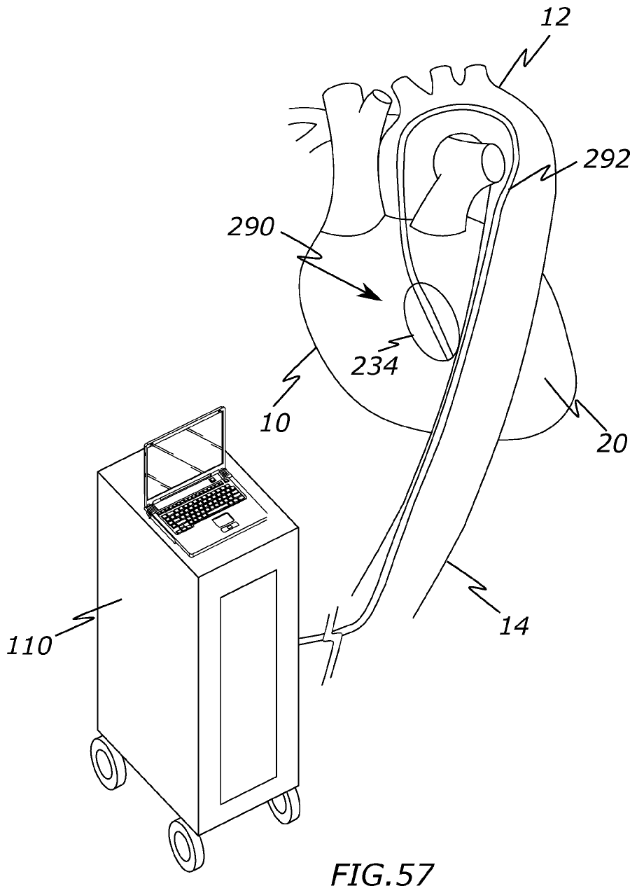

In another example, FIG. 57 illustrates only a counter pulsation balloon catheter 290. In this respect, the counter pulsation balloon catheter 290 alone (or with other catheters described in this specification) may be used. In one example, use of the counter pulsation balloon catheter 290 method includes positioning the counter pulsation balloon 234 with in the left ventricle, and inflating during diastole and deflating during systole, alone or with other devices described in this specification. This method is applicable to any embodiment with a counter pulsation balloon 234.

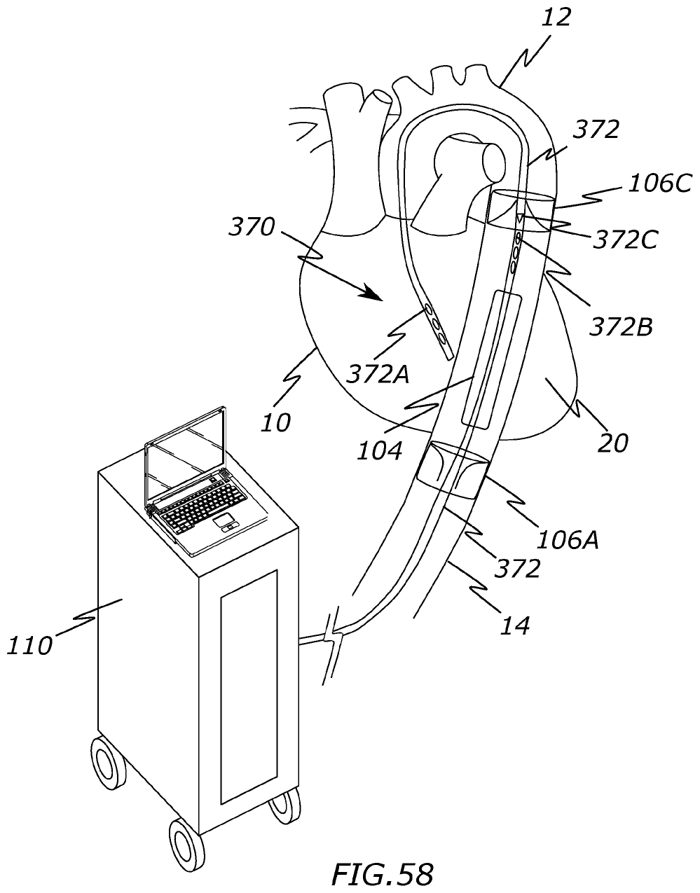

FIG. 58 illustrates one embodiment of catheter 370 that pumps blood from the left ventricle 20 into the aorta (e.g., descending aorta 14). The catheter 370 includes a distal portion that has one or a plurality of inlet apertures 372A that open into a lumen of the catheter body 372. When positioned in a patient, the lumen extends around the aortic arch 12 and then opens to outlet apertures 372B within the descending aorta 14.

In this embodiment, the blood is pumped in a similar manner with balloon pump 104 through the inlet apertures 372A, catheter lumen, and outlet 372B. In that regard, distal valve 106C is configured to open and close in the opposite direction of valve 106B, namely closed in an antegrade direction and open in a retrograde direction, though the other catheter elements function as previously discussed.

This allows blood to be pumped, via pumping balloon 104, directly from the left ventricle 20 and into the descending aorta 14, bypassing any vessels in the aortic arch 12. Specifically, the pumping balloon 104 inflates during diastole and deflates during systole. To assist in this blood flow, a one-way valve 372C (or alternately a toggle valve) may be included in the internal catheter lumen to allow antegrade blood flow through the catheter lumen but not retrograde blood flow. When the pumping balloon 104 deflates, it pulls blood through the outlet apertures 372B and antegrade through the descending aorta 14. Hence, the pumping balloon 104 pumps blood directly from the left ventricle 20 without “stealing” or pulling blood from vessels within the aortic arch 12 (e.g., cerebral and coronary arteries).

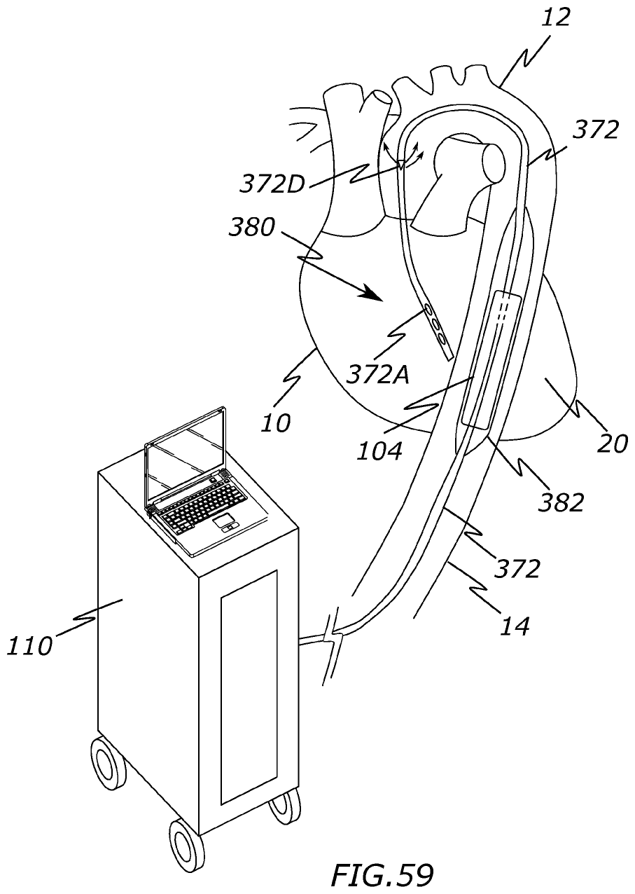

FIG. 59 illustrates another embodiment of a catheter 380 that pumps blood from the left ventricle 20 into an expandable closed lumen structure 382 and then back out at a distal toggle valve 372D. The closed lumen structure 382 is closed at its proximal and distal ends, connecting to the elongated catheter body 372 so as to form a blood impermeable chamber that surrounds the pumping balloon 104. As the pumping balloon 104 deflates, it pulls in blood from the inlet apertures 372A located within the left ventricle 20, into the internal blood passage of the catheter body 272, and then into the chamber of the closed lumen structure 382. When the pumping balloon 104 inflates, it pushes the blood within the chamber of the of the closed lumen structure 382 back through the lumen of the catheter body 272 until it reaches the toggle valve 372D positioned distally of the closed lumen structure 382. The toggle valve 372D is configured to open to an exterior of the catheter body 272 when blood flows in a retrograde direction (i.e., when the pumping balloon 104 is inflated) but otherwise allows passage through the lumen of blood flow in an antegrade direction. Hence, as the blood moving retrograde is pushed out into the aorta. The toggle valve 372D may be located within the ascending aorta, within the aortic arch, or within a top portion of the descending aorta.

In an alternate embodiment, the catheter 380 may not include the closed lumen structure 382. Hence, the pumping balloon 104 may pull blood through both the aorta and the lumen of the catheter.

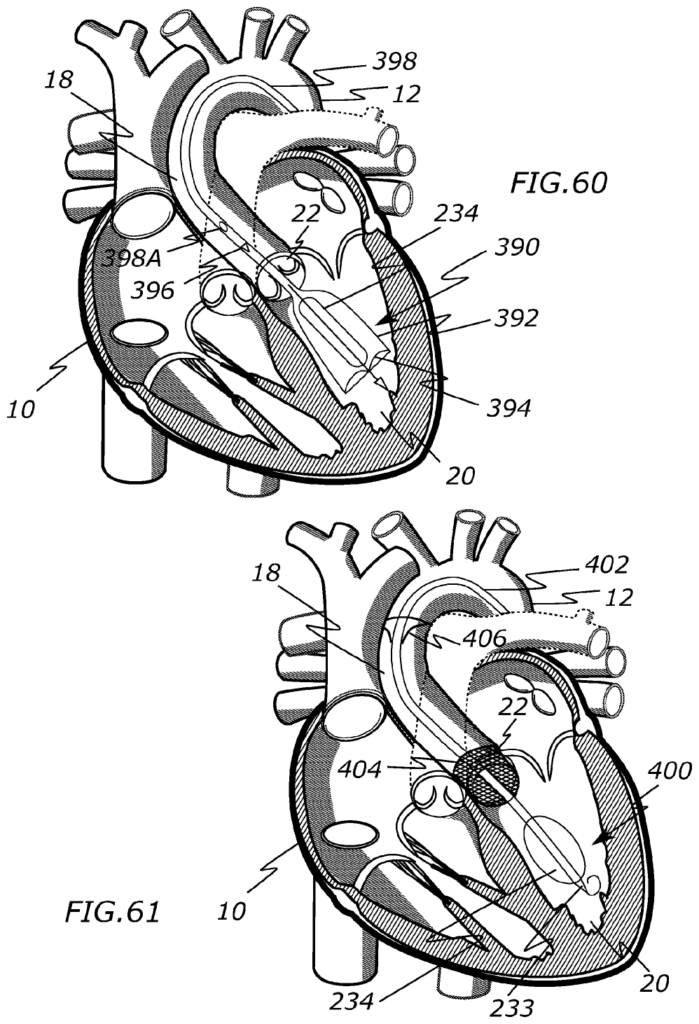

In some embodiments, an expandable structure may be placed around the counter pulsation balloon 234 to force blood from the left ventricle 20 into a passage of a catheter and out an outlet beyond the aortic valve 22. For example, FIG. 60 illustrates a catheter device 390 having an elongated body 398 with a counter pulsation balloon 234 located near its distal end and an expandable portion 392 surrounding the balloon 234. The expandable portion 392 may comprise a stent-like structure (e.g., braided tubular structure or laser cut tubular structure) that has an outer cover that limits or prevents passage of blood therethrough. This allows the expandable portion 392 to have a radially compressed configuration and a radially expanded configuration.

A proximal end of the expandable portion 392 may be fixed to the catheter body 398, optionally forming a conical end shape. A distal end of the expandable portion 392 has a larger diameter than the proximal end so as to allow blood flow into the interior of the expandable portion 392. The distal end may further include a one-way valve 394 (similar to previously described one-way valves) that allows blood into the interior of the expandable portion 392 but not out of the interior.

The interior of the expandable portion 392 connects to or is in communication with an internal passage within the catheter body 398 that opens outside of the catheter body 398 at outlet aperture 398A. This outlet aperture 398A may be positioned antegrade beyond the aortic valve 22 at a location within the aorta. Additionally, the passage within the catheter body 398 may include a second one way valve 396 that is configured to allow flow proximally in an antegrade direction but not distally in a retrograde direction.

The catheter body 398 may also include an inflation lumen between the proximal end and the balloon 234 to cause inflation of the balloon 234 at the desired time (e.g., inflation during diastole and deflation during systole via a control device 110).

In practice, the balloon 234 deflates, drawing in blood from the left ventricle 20, through one-way valve 394, and into the interior of the expandable portion 292. The second one-way valve 396 in the passage of the catheter body 398 prevents blood from being drawn into the passage from the aorta.

The balloon 234 is then inflated. The one-way valve 394 prevents blood from being pushed out into the left ventricle 20, forcing the blood in an antegrade direction through the internal passage of the catheter body 398, through the second one-way valve 396, and out the outlet aperture 398A into the aorta. Hence, blood may be pumped from the left ventricle 20, through the catheter body 398 and into the aorta.

The catheter 390 may be used alone or may be used with an intra-aortic balloon pump (and variations thereof) as previously described. The intra-aortic balloon may be on a separate catheter or may be incorporated on catheter 390.

In some embodiments that use a counter pulsation balloon 234, it may be desirable to increase the workable space within the left ventricle 20, since it may be difficult to include multiple components of the same or different catheters within only the left ventricle 20. One approach to increasing space may include holding open the aortic valve 22 and including a one way valve further down the ascending aorta 18. This effectively creates a larger space (namely both the left ventricle 20 and part of the ascending aorta 18) for a larger catheter or multiple catheters (e.g., a second ventricular assist catheter).

For example, FIG. 61 illustrates a counter pulsation balloon catheter 400 that includes a counter pulsation balloon 234 at its distal end which is configured in a manner previously discussed, as well as an expandable member 404 and downstream one-way valve 406. The expandable member 404 is positioned and sized such that it can be expanded within the aortic valve 22 and hold the valve open. The expandable member 404 may be composed of a tubular structure, such as a stent-like structure (e.g., braided wires or laser cut tube) that expands from a compressed configuration to an expanded configuration against the valve leaflets.

The one-way valve 406 may be positioned further antegrade in the ascending aorta, preferably prior to the aorta splitting off into other vessels. The one-way valve 406 is configured to allow blood flow antegrade but prevent blood flow retrograde, similar to the aortic valve 22. If another catheter, such as a stand-alone ventricular assist device (e.g., an Impella device) is also use, its distal end may be positioned within the enclosed area of the ascending aorta 18 and may not need to be positioned all the way into the left ventricle 20, thereby allowing more space for the counter pulsation balloon 234 to expand.

In any of the embodiments of this specification that utilize two or more catheters for simultaneous treatment, such as a counter pulsation balloon catheter and either a ventricular assist device or an intra-aortic balloon pump catheter, both catheters may be configured as rapid exchange catheters and used on the same guidewire.

In any of the embodiments of this specification that utilize two or more catheters for simultaneous treatment, such as a counter pulsation balloon catheter and either a ventricular assist device or an intra-aortic balloon pump catheter, a crimping mechanism may be included with at least one of the catheters to allow one catheter to be crimped to another prior to the procedure. This may allow the physician to determine a desirable distance or offset between ends of the catheters such that they reach and maintain a desired location within the patient, such as the left ventricle and the descending aorta. The crimping mechanism may include deformable metal portions extending from a first catheter that are configured to wrap around a second catheter.

Alternately, a first catheter may include one or a plurality of elastomeric rings or tubes that a second catheter can be placed through so that the two catheters can be aligned relative to each other.

Alternately, a first catheter may include a first magnet located on it and the second catheter may have a second magnet located on it and configured to attract to the first magnet. These magnets may allow the two catheters to be engaged with each other either before enter the patient or within the patient. Additionally, one or both magnets can be longitudinally moveable on their respective catheters to allow a physician to change their relative placement to each other.

In any of the embodiments of this specification that utilize one or more balloons, such as the intra-aortic balloon 104 or counter pulsation balloon 234, either a single balloon may be used or multiple balloons distributed along a length of their catheter may be used.

In some embodiments, it may be desirable to improve blood flow by placing a ventricular assist device within a coronary sinus of a patient and to pump blood flow through the coronary sinus and into the right atrium of a heart.

In some embodiments, a ventricular assist device (e.g., device 236) is described as pumping blood from the left ventricle 20 into the aorta. In such embodiments, it may be desirable to vary the flow rate of the pump (e.g., the speed of the internal impeller) in a way that there is a reduced pressure in the left ventricle in pressure in the left ventricle 20. This may be achieved via the control device controlling the speed of the pumping performed by the ventricular assist device, including increasing the pumping speed during systole and then decreasing or stopping pumping during diastole. Hence, the pressure within the left ventricle may be maintained relatively constant.

In one example, an algorithm may be used to determine a stroke volume of ventricle and periodically removing about ⅓ of the left ventricle volume when filled via the ventricular assist device. In a specific example, if the stroke volume is determined to be about 50 cc/beat and a heart rate was about 60 beats/min, 50 cc may be pumped every ⅓ second, 150 cc per second.

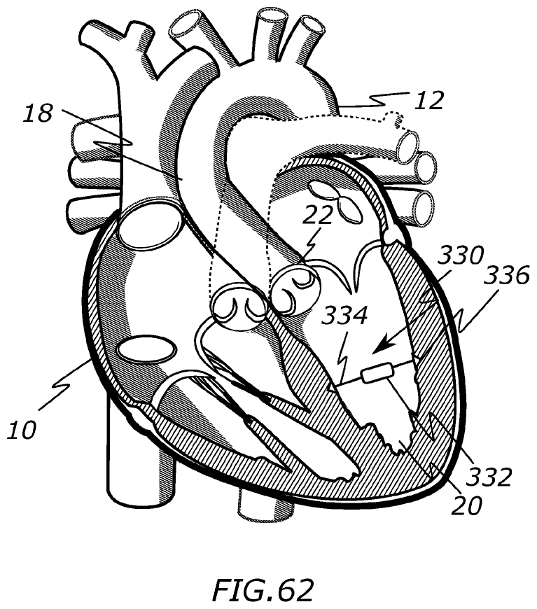

FIG. 62 illustrates another embodiment of a device configured to assist a heart 10 in pumping blood by mechanically moving portions of the heart 10 during a cardiac cycle. For example, FIG. 53 illustrates a heart movement device 330 comprising a motor assembly 332 with two or more tethers 334 that are connected to areas of the left ventricle 20 via anchors 336. The motor assembly 332 may include sensors (or can be in communication with external sensors) to determine when to activate during a cardiac cycle. At the appropriate time, the motor assembly 332 pulls on the two or more tethers 334, which decreases the amount of work performed by the heart muscle and/or increases the blood output of the heart 10.

The motor assembly 332 may include a motor, pulley, turnbuckle, and other mechanisms capable of pulling the tethers 334. The motor may be battery operated or externally powered. The sensors of the motor assembly 332 may include sensors to measure blood pressure, blood pH, blood salinity, tissue temperature, and electrical activity.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An intra-aortic blood pump, comprising:
an elongated catheter body;
a first pumping balloon connected to the catheter body and configured to inflate and deflate via an inflation lumen within the catheter body; and,
a first valve connected to the catheter body and configured to allow blood flow outside of the catheter body in a proximal direction and antegrade within a patient, and limit blood flow in a distal direction and retrograde within a patient.

2. The intra-aortic blood pump of claim 1, wherein the first valve is located distally of the first pumping balloon.

3. The intra-aortic blood pump of claim 1, wherein the first valve is located proximally of the first pumping balloon.

4. The intra-aortic blood pump of claim 1 further comprising a second valve connected to the catheter body and configured to allow blood flow outside of the catheter body in a proximal direction and antegrade within a patient, and limit blood flow in a distal direction and retrograde within a patient.

5. The intra-aortic blood pump of claim 4, wherein the first valve is positioned proximally of the first pumping balloon and the second balloon is positioned distally of the first pumping balloon.

6. The intra-aortic blood pump of claim 1, wherein the first valve comprises flaps.

7. The intra-aortic blood pump of claim 1, wherein the first valve comprises an occlusion balloon.

8. The intra-aortic blood pump of claim 1, wherein the first valve comprises a valve configured to open when inflated.

9. The intra-aortic blood pump of claim 8, wherein the first valve comprises an inflatable balloon within a framework configured to be biased to a closed, occluded configuration when the balloon is deflated and to change shape to an open, non-occluded configuration when the balloon is inflated.

10. The intra-aortic blood pump of claim 1, wherein the first valve comprises a concave shape formed of a plurality of struts and a flexible material connected to the plurality of struts.

11. The intra-aortic blood pump of claim 1, further comprising a radially expandable tubular member positioned around the first pumping balloon and connected to the first valve.

12. The intra-aortic blood pump of claim 11, wherein the tubular member has a decreased compliance relative to an aorta.

13. The intra-aortic blood pump of claim 11, further comprising a second valve connected to the tubular member.

14. The intra-aortic blood pump of claim 1, wherein the first valve is configured to also allow some retrograde backflow of blood within a range of about 5%-40%.

15. The intra-aortic blood pump of claim 1, further comprising a second pumping balloon configured for placement within an aortic arch, distal of the first pumping balloon.

16. The intra-aortic blood pump of claim 15, wherein the second pumping balloon is connected at a distal portion of the catheter body.

17. The intra-aortic blood pump of claim 16, further comprising a covered stent positioned over the first pumping balloon and the second pumping balloon.

18. The intra-aortic blood pump of claim 17, further comprising a second valve connected at a distal end of the covered stent.

19. The intra-aortic blood pump of claim 1, further comprising a counter pulsation balloon configured for placement within a left ventricle.

20. A method of pumping blood within a patient, comprising:
inflating an intra-aortic balloon within an aorta of a patient;
blocking blood flow in a retrograde direction of the aorta during the inflation of the intra-aortic balloon via a first valve.

21-55. (canceled)
